(19) United States
(12) Patent Application Publication
(10) Pub. No.: US 2017/0029454 A1
(43) Pub. Date: Feb. 2, 2017

(54) NKT CELL LIGANDS AND METHODS OF USE

(71) Applicants: THE SCRIPPS RESEARCH INSTITUTE, La Jolla, CA (US); BRIGHAM YOUNG UNVERSITY, Provo, UT (US)

(72) Inventors: Luc TEYTON, Del Mar, CA (US); Paul SAVAGE, Mapleton, UT (US)

(21) Appl. No.: 14/901,494

(22) PCT Filed: Jun. 27, 2014
(86) PCT No.: PCT/US2014/044684

§ 371 Date: Dec. 28, 2015

Related U.S. Application Data
(60) Provisional application No. 61/841,092, filed on Jun. 28, 2013.

(51) Int. Cl. C07H 15/10; G01N 33/50; A61K 39/00; A61K 39/12; A61K 39/02

(52) U.S. Cl. CPC C07H 15/10; A61K 39/12; A61K 39/02; A61K 39/0011; G01N 33/505

(57) ABSTRACT

Alpha-glycosylceramide compounds capable of activating NKT cells and compositions thereof are disclosed. Methods for activating NKT cells, methods of stimulating an immune response in a subject, and methods of treating cancer, infectious diseases, autoimmune diseases and disorders, or allergy diseases or disorders with the compounds and compositions are also disclosed.

This application is being filed on 27 Jun. 2014, as a PCT International patent application, and claims priority to U.S. Provisional Patent Application No. 61/841,092, filed Jun. 28, 2013, the disclosure of which is hereby incorporated by reference herein in its entirety.

### INTRODUCTION

Natural killer T cells (“NKT cells”) are a small population of innate-like memory/effector cells that express both natural killer (NK) receptors and a conserved, semi-invariant T cell receptor (TCR), (Vβ14-Jα18N/Vβ8 in mice and Vα14-Jα18N/Vβ11 in humans). NKT cells sit at the interface between innate and adaptive immunity and have been shown to be important for the coordination of T and B cell responses. For example, NKT cells have been implicated in suppression of autoimmunity and graft rejection, promotion of resistance to pathogens, and promotion of tumor immunity.

NKT cells are recruited very rapidly and transiently in the contact of all microbial aggressions to allow the maturation of dendritic cells (DC) and the recruitment of immune cells to the site of injury. The activation of NKT cells is believed to be dependent on the display of endogenous glycolipids by DCs in the context of CD1 MHC-like molecules.

NKT cells are capable of almost immediate responses leading to the hypothesis that endogenous ligands are either pre-made or quickly produced by an enzymatic modification that is tightly controlled to avoid persistent or overt activation and cell death and stunning. NKT cells respond with vigorous cytokine by releasing TH1-type cytokines, including IFN-γ and TNF, as well as TH2-type cytokines, including IL-4 and IL-13. Thus, NKT cells exhibit a dual function: they act as immunosuppressive cells via their production of TH2-type cytokines; and also act as immune promoters to enhance cell-mediated immunity via the production of TH1-type cytokines.

NKT cells recognize foreign and self lipid antigens presented by the CD1d member of the family of β2 microglobulin-associated molecules. A variety of lipids with different structures have been shown to bind CD1d molecules in a unique manner that accommodates a fatty acid chain in each of the two hydrophobic binding pockets (A′ and F) of the CD1d molecule. Lipid species capable of binding CD1d molecules include mycolic acids, diacylglycerols, sphingolipids, polyisoprenoids, lipopeptides, phosphomycoketides and small hydrophobic compounds. The evolutionary conservation of NKT cells is striking, as mouse NKT cells recognize human CD1d plus glycolipid antigen and vice versa.

A large number of potential endogenous ligand candidates have been proposed over the years, all capable of activating NKT cells in vitro and/or in vivo (Zhou et al., 2004, Science, 306:1786; Brennan et al., 2011, Nature Immunology, 12: 1202; Facciotti et al., 2012, Nature Immunology, 13:474). However, the chemistry of these potential candidates has proven difficult to study due to the lack of sensitivity of lipid analytical methods. Glycosylceramides are believed to be endogenous NKT ligands. Chemical and biochemical studies have established that glycosylceramides are β anomers in mammalian species. These conclusions have been reinforced by the identification of only one glucosylceramide synthase and one galactosylceramide synthase in mammalian genomes, both β-transferases. Accordingly, it has been proposed that β-glycosylceramides (βGluCer) are the natural endogenous ligand of NKT cells and synthetic preparations of C12 and C24:1 βGluCer have been shown to be strong activators of type 1 NKT cells (Brennan et al., 2011, Nature Immunology, 12: 1202).

### SUMMARY OF THE INVENTION

Utilizing the specificity of immunological assays in combination with enzymatic assays, natural endogenous ligands for NKT cells have been identified and characterized. Surprisingly, these stimulatory NKT agonists are not β-anomers of glycosylceramides but α-linked monoglycosylceramides, a class of glycolipids thought to be absent from mammalian cells. The compounds of the disclosure provide a basis for manipulating NKT cell production and numbers, elucidating the function of NKT cells in multiple contexts, such as cancer, infectious diseases, and autoimmune disorders, and provide novel therapeutics and methods for treating these diseases and disorders.

In one aspect, the disclosure provides glycolipid compounds represented by formula I:

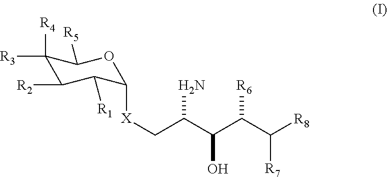

wherein:
X is O, S, or CH₂;
R₁, is —OR₉, wherein R₉ is —H, —SO₃H, or a pharmaceutically acceptable salt;
R₂ is —OH, —SO₃H, —OSO₃H, —PO₄, —PO₄H, —COOH, or a pharmaceutically acceptable salt;
R₃ is —H if R₄ is —OR₉ or R₃ is —OR₉ if R₄ is —H;
R₅ is —C(O)R₆ wherein R₆ is —OH, —OSO₃H, or a pharmaceutically acceptable salt thereof or —CH₂OR₉;
R₆ is —H, —OR₉, or forms a double bond with R₇;
R₇ is —H or forms a double bond with R₆; and
R₈ is a saturated or unsaturated hydrocarbon having from about 5 to about 15 carbons.

In another aspect, the disclosure provides glycolipid compounds represented by formula II:

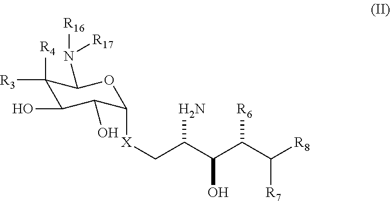

wherein:
X is O, S, or CH₂;
R₁₆ is selected from:
   (i) C(O) R₁₃;
   (ii) C(R₁₃)R₁₄, wherein R₁₄ is —H and R₂ forms a double bond between nitrogen and the carbon to which R₁₄ is attached;
   (iii) C(R₁₃)R₁₄(R₁₅), wherein R₁₄ is H or R₁₃ and R₁₅ is —H or R₁₃; or
   (iv) SO₂R₁₃;
      wherein R₁₃ is halo; hydroxy, OR₉; OR₁₀; amino, NHR₉; N(R₉)₂; NHR₁₀; N(R₁₀)₂; aralkylamino; or C₁-C₁₂ alkyl optionally substituted with halo, hydroxyl, oxo, nitro, OR₉, OR₁₀, acyloxy, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₉, NHSO₂R₁₀, sulfate, phosphate, cyano, carboxyl, C(O)R₉, C(O)R₁₀, C(O)OR₉, C(O)NH₂, C(O)NHR₉, C(O)N(R₉)₂, C₃-C₁₀ cycloalkyl containing 0-3 R₁₁, C₃-C₁₀ heterocycyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl containing 0-3 R₁₂, or heteroaryl containing 0-3 R₁₂; or C₃-C₁₀ cycloalky, C₃-C₁₀ heterocyclyl, C₅-C₁₀ cycloalkenyl, or C₅-C₁₀ heterocycloalkenyl optionally substituted with one or more halo hydroxyl, oxo, OR₉, OR₁₀, acyloxy, nitro, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₉, NHSO₂R₁₀, sulfate, phosphate, cyano, carboxyl, C(O)R₉, C(O)R₁₀, C(O)OR₉, C(O)NH₂, C(O)NHR₁₀, C(O)N(R₁₀)₂, alkyl, haloalkyl, C₃-C₁₀ cycloalkyl containing 0-3 R₁₁, C₃-C₁₀ heterocyclyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl heteroaryl containing 0-3 R₁₂, or C₆-C₂₀ heteroaryl containing 0-3 R₁₂; or C₂-C₆ alkenyl, C₂-C₆ alkynyl, aryl, or heteroaryl optionally substituted with one or more halo, hydroxyl, OR₉, OR₁₀, acyloxy, nitro, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₁₀, sulfate, phosphate, cyano, carboxyl, C(O)R₉, C(O)R₁₀, C(O)OR₉, C(O)NH₂, C(O)NHR₉, C(O)N(R₉)₂, alkyl, haloalkyl, C₃-C₁₀ cycloalkyl containing 0-3 R₁₁, C₃-C₁₀ heterocycyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl containing 0-3 R₁₂, or C₆-C₂₀ heteroaryl containing 0-3 R₁₂;
R₁₇ is —H or C₁-C₆ alkyl;
R₃ is —H if R4 is —OH, or R₃ is —OH if R₄ is —H;
R₆ is —OH or forms a double bond with R₇;
R₇ is —H or forms a double bond with R₆;
R₈ is a saturated or unsaturated hydrocarbon having from about 5 to about 15 carbons;
each R₉ is independently a C₁-C₂₀ alkyl optionally substituted with halo, hydroxyl, alkoxy, amino, alkylamino, dialkylamino, sulfate, or phosphate;
each R₁₀ is independently an aryl optionally substituted with halo, haloalkyl, hydroxyl, alkoxy, nitro, amino, alkylamino, dialkylamino, sulfate, or phosphate;
each R₁₁ is independently halo, haloalkyl, hydroxyl, alkoxy, oxo, amino, alkylamino, dialkylamino, sulfate, or phosphate; and
each R₁₂ is independently halo, haloalkyl, hydroxyl, alkoxy, nitro, amino, alkylamino, dialkylamino, sulfate, or phosphate.

In another aspect, the disclosure provides glycolipid compounds represented by formula III:

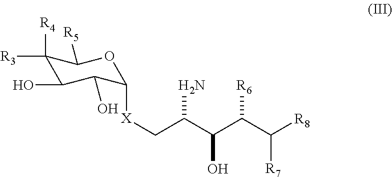

wherein:
X is O, S, or CH₂;
R₃ is —H if R₄ is —OH, or R₃ is —OH if R₄ is —H;
R₅ is —SR₁₅ or —OR₁₅;
   wherein R₁₅ is C₁-C₁₂ alkyl optionally substituted with halo, hydroxyl, oxo, nitro, OR₉, OR₁₀, acyloxy, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₉, NHSO₂R₁₀, sulfate, phosphate, cyano, carboxyl, C(O)R₉, C(O)R₁₀, C(O)OR₉, C(O)NH₂, C(O)NHR₉, C(O)N(R₉)₂, C₃-C₁₀ cycloalkyl containing 0-3 R₁₁, C₃-C₁₀ heterocycyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl containing 0-3 R₁₂, or heteroaryl containing 0-3 R₁₂; or C₃-C₁₀ cycloalky or C₅-C₁₀ cycloalkenyl optionally substituted with one or more halo hydroxyl, oxo, OR₉, OR₁₀, acyloxy, nitro, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₉, NHSO₂R₁₀, sulfate, phosphate, cyano, carboxyl, C(O)R₉, C(O)R₁₀, C(O)OR₉, C(O)NH₂, C(O)NHR₁₀, C(O)N(R₁₀)₂, alkyl, haloalkyl, C₃-C₁₀ cycloalkyl containing 0-3 R₁₁, C₃-C₁₀ heterocyclyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl heteroaryl containing 0-3 R₁₂, or C₆-C₂₀ heteroaryl containing 0-3 R₁₂; or C₂-C₆ alkenyl, C₂-C₆ alkynyl, or aryl, optionally substituted with one or more halo, hydroxyl, OR₉, OR₁₀, acyloxy, nitro, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₁₀, sulfate, phosphate, cyano, carboxyl, C(O)R₉, C(O)R₁₀, C(O)OR₉, C(O)NH₂, C(O)NHR₉, C(O)N(R₉)₂, alkyl, haloalkyl, C₃-C₁₀ cycloalkyl containing 0-3 R₁₁, C₃-C₁₀ heterocycyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl containing 0-3 R₁₂, or C₆-C₂₀ heteroaryl containing 0-3 R₁₂;
R₆ is —OH or forms a double bond with R₇;
R₇ is —H or forms a double bond with R₆;
R₈ is a saturated or unsaturated hydrocarbon having from about 5 to about 15 carbons;
each R₉ is independently a C₁-C₂₀ alkyl optionally substituted with halo, hydroxyl, alkoxy, amino, alkylamino, dialkylamino, sulfate, or phosphate;
each R₁₀ is independently an aryl optionally substituted with halo, haloalkyl, hydroxyl, alkoxy, nitro, amino, alkylamino, dialkylamino, sulfate, or phosphate;
each R₁₁ is independently halo, haloalkyl, hydroxyl, alkoxy, oxo, amino, alkylamino, dialkylamino, sulfate, or phosphate; and
each R₁₂ is independently halo, haloalkyl, hydroxyl, alkoxy, nitro, amino, alkylamino, dialkylamino, sulfate, or phosphate.

Compositions comprising one or more compounds of formula I, formula II, formula III, or a combination thereof are also provided. The compositions of the disclosure can include a physiological acceptable vehicle. In some embodiments, the composition further includes an antigen, such as a tumor antigen, viral antigen, or microbial antigen. In some embodiments, the composition is formulated as a vaccine.

In another aspect, methods of activating an NKT cell comprising contacting the NKT cell with the compound of formula (I) or formula (II) in the presence of CD1d are provided. The CD1d can be in soluble form, such as a CD1d tetramer, or CD1d expressed on the surface of a cell, such as an antigen presenting cell.

In yet another aspect, methods of stimulating an immune response in a subject are provided. In some embodiments, the methods include administering to the subject an effective amount of the compound of formula I, formula II, formula III, or a combination thereof. In other embodiments, the methods include administering to the subject an inhibitor of ceramidase or α-glycosidase to induce and/or enhance expression of α-glycosylceramides by antigen presenting cells. Alternatively, the method of stimulating an immune response in a subject comprises a step of administering to the subject a population of NKT cells activated by contacting the NKT cells with antigen presenting cells comprising CD1d loaded with compounds of the disclosure or antigen presenting cells treated with an inhibitor of ceramidase or α-glycosidase to induce and/or enhance expression of α-glycosylceramides by the antigen presenting cells. Alternatively, the method of stimulating an immune response in a subject comprises administering to the subject a population of CD1+ antigen presenting cells contacted with a compound of the disclosure or treated with an inhibitor of ceramidase or α-glycosidase to induce and/or enhance expression of α-glycosylceramides by the antigen presenting cells.

In yet another aspect, methods of modulating NKT cell activation are provided. In embodiments, an antibody that binds α-glycosylceramides is administered to reduce or block activation of NKT cells by α-glycosylceramides. The methods can be used to treat diseases and disorders, such as autoimmune or allergy diseases or disorders, in which a reduction in NKT cell activation is desirable.

In yet another aspect, methods of screening and identifying NKT cells agonists are provided. The methods generally include treating antigen presenting cells with a candidate inhibitor of ceramidase or a α-glycosidase, contacting NKT cells with the treated antigen presenting cells, and determining the activation of the contacted NKT cells wherein an increase in NKT cell activation relative to control NKT cells indicates the candidate inhibitor is an NKT cell agonist.

### BRIEF DESCRIPTION OF THE DRAWINGS

FIG. 1 depicts a first embodiment of a synthesis scheme according to the disclosure.

FIG. 2 depicts a second embodiment of a synthesis scheme according to the disclosure.

FIGS. 3A and 3B depict a schematic representation of the synthetic and catabolic pathways of monoglycosyl and monolysoglycosylceramides.

FIG. 4A depicts the IL-2 production of Vα14 expressing DN32.D3 NKT cells tested after a 24-hour exposure to increasing numbers of RBL-CD1 cells in the presence of L363 (open circles) or control (filled circles) antibody (10 μg/ml).

FIG. 4B depicts the non-Vα14 NKT cell hybridoma TBA.7 tested under similar conditions as in FIG. 4A.

FIGS. 4C and 4D depict stimulation of DN32.D3 cells (4C) and TBA.7 cells (4D) tested against RBL-CD1 (filled circles) or RBL-CD1 SAP^{-/-} cells in which saposin expression was knocked down by interfering RNAs.

FIG. 4E depicts the stimulatory activity of WT thymocytes towards DN32.D3 cells tested in the presence of control (filled circles) or L363 (open circles) antibody (20 μg/ml).

FIG. 4F depicts IL-2 production of DN32.D3 cells in the presence of control (filled circles) or L363 (open circles) antibody (10 μg/ml). The DN32.D3 cells were stimulated with 2×10⁴ DC3.2 cells treated for 16 hours with increasing concentrations of LPS.

FIG. 5 depicts the predicted L363 binding to glycosylceramides.

FIG. 6 depicts separation and functional analysis of commercial β-glucosylceramide 24:1.

FIG. 7A depicts TLC analysis (right panel) of β-glucosylceramide digested with recombinant GBA for 2 hours at 37° C. and its ability to stimulate DN32.D3 NKT cells (left panel) when presented by WT splenocytes (10⁵ cells/well). Stimulatory activity was not changed after (squares) as compared to before (circles) digestion.

FIG. 7B shows that the stimulatory activity of commercial β-glucosylceramide is blocked by L363 (diamondsX) (10 μg/ml) and 20H2 (triangles) (5 μg/ml) but not control (squares) antibodies.

FIG. 8 shows the binding of L363 antibody to various α and β anomers of glycosylceramides as measured by surface plasmon resonance. Single cycle analysis was performed on CM5 chips using 250-1000 RUs of immobilized antibody and increasing concentrations of CD1-lipid complexes.

FIG. 9 shows the lipid content of L363 and L317 antibody immunoprecipitations from DC3.2 and RBL-CD1 cells (2×10⁹ cells) analyzed by LC-Multiple Reaction Monitoring (MRM) mass spectrometry.

FIGS. 10A-D show induction NKT ligands on DC3.2 cells treated with recombinant TNFα. Inhibition of α-glycosidase activity with the identified inhibitors (GLAi, GAAi, or GLAi+GAAi) induced or increased stimulation of NKT cells.

FIGS. 11A-C show in the context of TNFα stimulation of the DC3.2 cells, the inhibitors GLAi and GAAi have similar effects as in FIG. 10 and did not increase stimulation of NKT cells.

FIGS. 12A and 12B depict the separation of glycosyl (12A) and lysoglycosylceramides (12B) by high performance TLC before and after digestion with recombinant GLA. Gal: galactosyl-, glu: glucosyl-).

FIGS. 13A and 13B depict stimulatory ability tests of samples from α-galactosyl (13A) and α-psychosine (13B), towards DN32.D3 NKT cells before (open circles) and after (filled circles) digestion with recombinant GLA. DC3.2 cells (20,000 cells/well) were used as antigen presenting cells.

FIGS. 14A-E depict the IL-2 production of DN32.D3 NKT cells stimulated with DC3.2 cells differentiated with LPS and treated with inhibitors of α-glycosidases (GLAi and/or GAAi), 1-deoxygalactonojirimycin (0.5 μM) and 1-deoxygluconojirimycin (2.0 μM), respectively, or ceramidase inhibitors (NAAA/ASAHLi (20 μM) or AC/ASAH1i, carmofur (1.0 μM)) for 24 hours. Control (open circles) is included in each panel for comparison with inhibitors (filled circles).

FIGS. 15A-E depict the IL-2 production DN32.D3 NKT cells without or in the presence of the same inhibitors of glycosidases and ceramidases as used in FIGS. 14A-E using thymocytes as antigen presenting cells. Control (open circles) is included in each panel for comparison with inhibitors (filled circles).

FIGS. 16A and 16B depict titration of DN32.D3 stimulation inhibition by L363 antibody when thymocytes (16A) or RBL-CD1 cells (16B) were used as antigen presenting cells.

FIG. 16C shows percentage inhibition plotted as percentage of maximal response (100%) for RBL-CD1 (black symbols) and thymocytes (open circles) of the samples of FIGS. 16A and 16B.

FIG. 17 shows day 14.5 thymic lobes cultured for 18 days in the presence of antibody 14.4.4s, L363, and 20H2, respectively, and stained with CD d/Empty or CD1d/PBS-57 tetramers.

### DETAILED DESCRIPTION

The biosynthetic pathways of glycolipids have been previously described in the context of enzymatic deficiencies that lead to inherited human diseases of the nervous system (Schulze and Sandhoff, 2011, Lysosomal lipid storage diseases, Cold Spring Harb. Perspect. Biol., 3; Wennekes et al., 2009, Angew Chem. Int. Ed. Engl., 48:8848). Using classical biochemical methods, a map of enzymes, their substrates and their products was produced and this map over time has become accepted. However, all lipid analytical methods lack sensitivity and these methods are not capable of detecting contaminations below 0.5-1% in natural or synthetic preparations of lipids and glycolipids (Meisen et al., 2011, Biochimica et biophysica acta, 1811:87). This lack in sensitivity of lipid analytical methods has hampered the identification of immunologically relevant lipid species, a family of antigens that is presented by the MHC-like molecules called CD1 (Bendelac et al., 2007, Annual Rev. Immunol., 25:297).

NKT cells make up a small population of regulatory T cells that sits at the interface between innate and adaptive immunities and is critical for the coordination of T and B cell responses (Bendelac et al., 2007, Annual Rev. Immunol., 25:297). As currently understood, NKT cells are recruited very rapidly and transiently in the context of all microbial aggressions to allow the maturation of dendritic cells (DC) and the recruitment of immune cells at the site of injury. The activation of NKT cells is believed to be dependent on the display of endogenous glycolipids by DC in the context of CD1 MHC-like molecules. NKT cells are capable of almost immediate responses leading to the hypothesis that endogenous ligands are either pre-made or quickly produced by an enzymatic modification that is tightly controlled to avoid persistent or overt activation and cell death or stunning (Wilson et al., 2003, Proceedings of the National Academy of Sciences of the United States of America 100:10913). A large number of potential candidates have been proposed over the years, all capable of activating NKT cells in vitro and/or in vivo (Zhou et al., 2004, Science, 306:1786; Brennan et al., 2011, Nature Immunology, 12: 1202; Facciotti et al., 2012, Nature Immunology, 13:474). However, the chemistry of these potential candidates has proven difficult due to the lack of sensitivity of lipid analytical methods.

Biological assays are exquisitely sensitive to low levels of otherwise unmeasurable molecules. Utilizing the specificity of immunological assays employing T and B lymphocytes in combination with the specificity of enzymatic assays employing catabolic enzymes of the sphingolipid pathway, glycolipids capable of triggering the activation of Natural Killer T cells (NKT cells) have been identified and characterized. These stimulatory NKT agonists were surprisingly alpha-linked monoglycosylceramides, a class of glycolipids that was thought to be absent from mammalian cells as the only two glycosylceramide synthases (glucosylceramide synthase (GCS) and ceramide galactosyl transferase (CGT)) are thought to be inverting glycosyltransferases which, through a S_{N}2-like ligation, transfer α-glucose and α-galactose from UDP-sugar moieties to a β anomeric position onto a ceramide (Lairson et al., 2008, Annual Rev. Biochem., 77:521). The α anomeric compounds of the disclosure provide a basis for manipulating NKT cell production and numbers, elucidating the function of NKT cells in multiple contexts, such as cancer, infectious diseases, and autoimmune disorders, and provide novel therapeutics for treating these diseases and disorders.

### DEFINITIONS

As used in this specification and the appended claims, the singular forms “a,” “an,” and “the” include plural referents unless the content clearly dictates otherwise. Thus, for example, reference to a composition containing “a glycolipid” includes a mixture of two or more glycolipids. It should also be noted that the term “or” is generally employed in its sense including “and/or” unless the content clearly dictates otherwise.

The term “glycolipid” refers to any compound containing one or more monosaccharide residues (“glyco” portion) bound by a glycosidic linkage to a hydrophobic moiety such as an acylglycerol, a sphingoid, a ceramide (N-acylsphingoid) or a prenyl phosphate (“lipid” portion). In particular embodiments, one or more saccharides are bound to a ceramide moiety.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it.

The terms “arylarkyl” or “aralkyl” refer to an alkyl moiety in which an alkyl hydrogen atom is replaced by an aryl group, for example benzyl or 9-fluorenyl groups.

The term “alkylamino” and “dialkylamino” refer to —NH(alkyl) and —NH(alkyl)₂ radicals respectively.

The term “alkoxy” refers to an —O-alkyl radical.

The term “mercapto” refers to an SH radical.

The term “thioalkoxy” refers to an —S-alkyl radical.

The term “aryl” refers to an aromatic moncyclic, bicyclic, or tricyclic hydrocarbon ring system, wherein any ring atom capable of substitution can be substituted by a substituent, such as, but not limited to, phenyl, naphthyl, and anthracenyl.

The term “cycloalkyl” as employed herein includes saturated cyclic, bicyclic, tricyclic, or polycyclic hydrocarbon groups having 3 to 12 carbons, wherein any ring atom capable of substitution can be substituted by a substituent. Examples of cycloalkyl moieties include, but are not limited to, cyclohexyl and adamantyl.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom capable of substitution can be substituted by a substituent.

The term “cycloalkenyl” as employed herein includes partially unsaturated, nonaromatic, cyclic, bicyclic, tricyclic, or polycyclic hydrocarbon groups having 5 to 12 carbons, preferably 5 to 8 carbons, wherein any ring atom capable of substitution can be substituted by a substituent. Examples of cycloalkyl moieties include, but are not limited to cyclohexenyl, cyclohexadienyl, or norbornenyl.

The term “heterocycloalkenyl” refers to a partially saturated, nonaromatic 5-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom capable of substitution can be substituted by a substituent.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom capable of substitution can be substituted by a substituent.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Suitable substituents include, without limitation, alkyl, alkenyl, alkynyl, alkoxy, halo, hydroxy, cyano, nitro, amino, SO₃H, sulfate, phosphate, perfluoroalkyl, perfluoroalkoxy, methylenedioxy, ethylenedioxy, carboxyl, oxo, thioxo, imino (alkyl, aryl, aralkyl), S(O)ₙalkyl (where n is 0-2), S(O)ₙaryl (where n is 0-2), S(O)ₙ heteroaryl (where n is 0-2), S(O)ₙheterocyclyl (where n is 0-2), amine (mono-, di-, alkyl, cycloalkyl, aralkyl, heteroaralkyl, and combinations thereof), ester (alkyl, aralkyl, heteroaralkyl), amide (mono-, di-, alkyl, aralkyl, heteroaralkyl, and combinations thereof), sulfonamide (mono-, di-, alkyl, aralkyl, heteroaralkyl, and combinations thereof), unsubstituted aryl, unsubstituted heteroaryl, unsubstituted heterocyclyl, and unsubstituted cycloalkyl. In one aspect, the substituents on a group are independently any one single, or any subset of the aforementioned substituents.

The term “antigen presenting cell” or “APC” refers to a cell capable of presenting antigen to NKT cells. Antigen presenting cells are generally CD1d+. Examples of antigen presenting cells include dendritic cells, macrophages, thymocytes, B cells, and Ito cells.

### Compounds

α-psychosine, α-gluco-psychosine, and derivative compounds thereof have been found to be potent agonists of NKT cells. As such, these compounds can enhance an immune response in a subject. Conversely, antagonists of these compounds can be used to modulate an immune response in a subject. Because the compounds of the disclosure are endogenous compounds or derivative compounds thereof, the likelihood of side effects is reduced in comparison to exogenous molecules. Derivatives of α-psychosine or α-gluco-psychine can be modified, for example, to introduce properties suitable for in vivo delivery and/or to modulate the NKT cell stimulatory activity of the compounds. Derivative compounds can include modifications to the ceramide head group, the carbohydrate, and/or sphingosine side chain. α-psychosine, α-gluco-psychine, and derivative compounds thereof that exhibit NKT cell agonist activity are collectively referred to herein as “NKT cell agonist compounds.”

In one aspect, compounds of the disclosure are glycolipids represented by formula I:

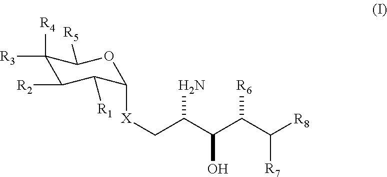

wherein:
X is O, S, or CH₂;
R₁, is —OR₉, wherein R₉ is —H, —SO₃H, or a pharmaceutically acceptable salt;
R₂ is —OH, —SO₃H, —OSO₃H, —PO₄, —PO₄H, —COOH, or a pharmaceutically acceptable salt;
R₃ is —H if R₄ is —OR₉ or R₃ is —OR₉ if R₄ is —H;
R₅ is —C(O)R₆ wherein R₆ is —OH, —OSO₃H, or a pharmaceutically acceptable salt thereof or —CH₂OR₉;
R₆ is —H, —OR₉, or forms a double bond with R₇;
R₇ is —H or forms a double bond with R₆; and
R₈ is a saturated or unsaturated hydrocarbon having from about 5 to about 15 carbons.

In some embodiments, compounds of the invention are glycolipids represented by formula I, shown below:

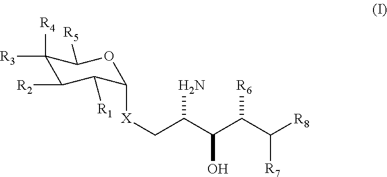

wherein:
X is O, S, or CH₂;
R₁, is —OR₉, wherein R₉ is —H, —SO₃H, or a pharmaceutically acceptable salt;
R₂ is —OH, —SO₃H, —OSO₃H, —PO₄, —PO₄H, —COOH, or a pharmaceutically acceptable salt;
R₃ is —H if R₄ is —OR₉ or R₃ is —OR₉ if R₄ is —H;
R₅ is —C(O)R₆ wherein R₆ is —OH, —OSO₃H, or a pharmaceutically acceptable salt thereof or —CH₂OR₉;
R₆ is —H, —OR₉, or forms a double bond with R₇;
R₇ is —H or forms a double bond with R₆; and
R₈ is a saturated or unsaturated hydrocarbon having from about 5 to about 15 carbons;
   with the proviso that the compound is not

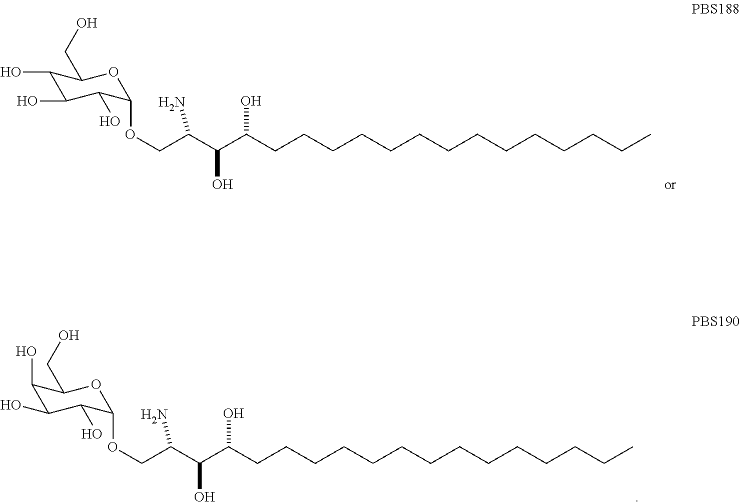 [SMILES: CCCCCCCCCCCCCC[C@@H](O)[C@@H](O)[C@@H](N)COC1OC(CO)C(O)C(O)C1O ; CCCCCCCCCCCCCC[C@@H](O)[C@@H](O)[C@@H](N)COC1OC(CO)C(O)C(O)C1O]

In some embodiments, R₁, R₂, and R₆ are OH, R₃ is —H if R₄ is —OH or R₃ is —OH if R₄ is —H, R₅ is —CH₂OH, R₆ is —H, —OH or forms a double bond with R₇, and R₈ is a saturated or unsaturated hydrocarbon having from about 5 to about 15 carbons.

In some embodiments, X is O, R₁, R₂, and R₆ are OH, R₃ is —H if R₄ is —OH or R₃ is —OH if R₄ is —H, R₅ is —CH₂OH, R₆ is —H, —OH or forms a double bond with R₇, and R₈ is a saturated or unsaturated hydrocarbon having from about 5 to about 15 carbons. Examples include but are not limited to

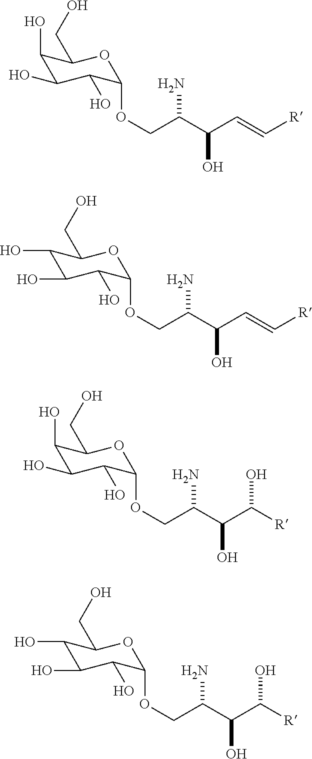

wherein R′ is a saturated or unsaturated hydrocarbon having from about 5 to about 15 carbons. Additional examples include but are not limited to

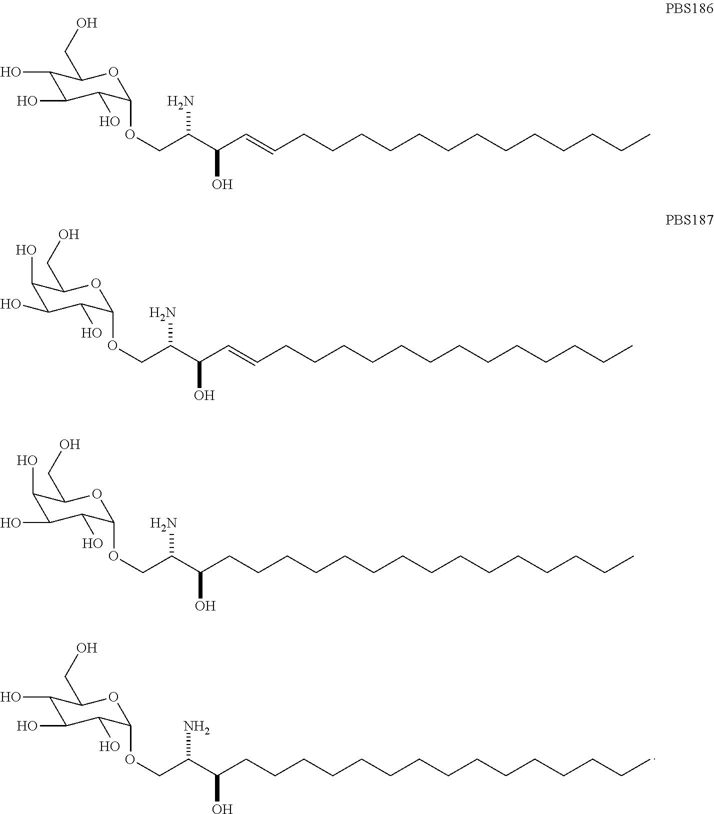 [SMILES: CCCCCCCCCCCCC/C=C/[C@@H](O)[C@@H](N)COC1OC(CO)C(O)C(O)C1O ; CCCCCCCCCCCCC/C=C/[C@@H](O)[C@@H](N)COC1OC(CO)C(O)C(O)C1O ; CCCCCCCCCCCCCCC[C@@H](O)[C@@H](N)COC1OC(CO)C(O)C(O)C1O ; CCCCCCCCCCCCCCC[C@@H](O)[C@@H](N)COC1OC(CO)C(O)C(O)C1O]

In some embodiments of formula I, R₁, R₂, and R₆ are OH, R₃ is —H if R₄ is —OH or R₃ is —OH if R₄ is —H, R₅ is —COOH, R₆ is —OH or forms a double bond with R₇, and R₈ is a saturated or unsaturated hydrocarbon having from about 5 to about 15 carbons.

In some embodiments of formula I, X is O, R₁, R₂, and R₆ are OH, R₃ is —H if R₄ is —OH or R₃ is —OH if R₄ is —H, R₅ is —COOH, R₆ is —OH or forms a double bond with R₇, and R₈ is a saturated or unsaturated hydrocarbon having from about 5 to about 15 carbons. Examples include but are not limited to

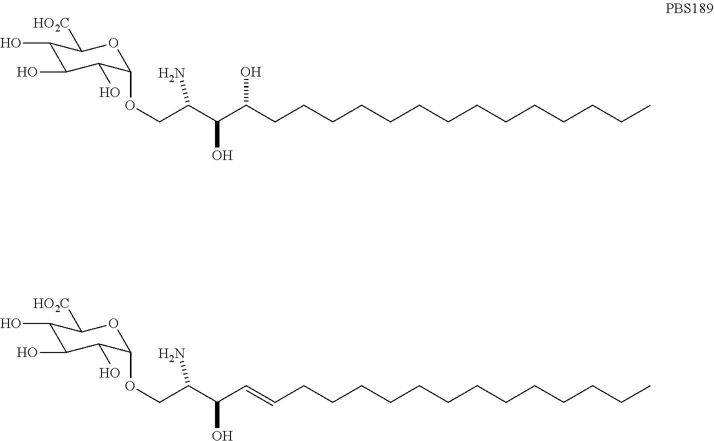 [SMILES: CCCCCCCCCCCCCC[C@@H](O)[C@@H](O)[C@@H](N)COC1OC(C(=O)O)C(O)C(O)C1O ; CCCCCCCCCCCCC/C=C/[C@@H](O)[C@@H](N)COC1OC(C(=O)O)C(O)C(O)C1O]

In some embodiments, the compound of formula (I) is represented by one of the following structures

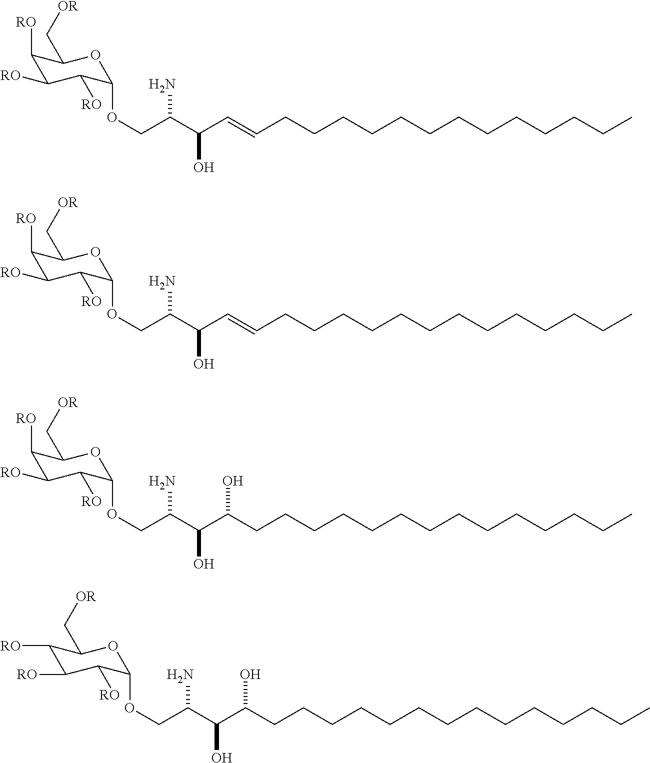

wherein R is independently —H, —OSO₃, or a pharmaceutically acceptable salt.

In another aspect, compounds of the disclosure are glycolipids represented by formula II:

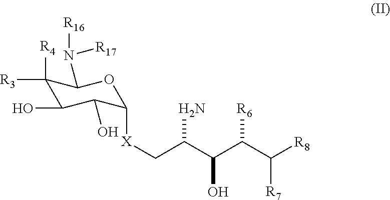

wherein:
X is O, S, or CH₂;
R₁₆ is selected from:
   (i) C(O)R₁₃;
   (ii) C(R₁₃)R₁₄, wherein R₁₄ is —H and R₂ forms a double bond between nitrogen and the carbon to which R₁₄ is attached;
   (iii) C(R₁₃)R₁₄(R₁₅), wherein R₁₄ is H or R₁₃ and R₁₅ is —H or R₁₃; or
   (iv) SO₂R₁₃;
      wherein R₁₃ is halo; hydroxy, OR₉; OR₁₀; amino, NHR₉; N(R₉)₂; NHR₁₀; N(R₁₀)₂; aralkylamino; or C₁-C₁₂ alkyl optionally substituted with halo, hydroxyl, oxo, nitro, OR₉, OR₁₀, acyloxy, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₉, NHSO₂R₁₀, sulfate, phosphate, cyano, carboxyl, C(O)R₉, C(O)R₁₀, C(O)OR₉, C(O)NH₂, C(O)NHR₉, C(O)N(R₉)₂, C₃-C₁₀ cycloalkyl containing 0-3 R₁₁, C₃-C₁₀ heterocycyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl containing 0-3 R₁₂, or heteroaryl containing 0-3 R₁₂; or C₃-C₁₀ cycloalky, C₃-C₁₀ heterocyclyl, C₅-C₁₀ cycloalkenyl, or C₅-C₁₀ heterocycloalkenyl optionally substituted with one or more halo hydroxyl, oxo, OR₉, OR₁₀, acyloxy, nitro, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₉, NHSO₂R₁₀, sulfate, phosphate, cyano, carboxyl, C(O)R₉, C(O)R₁₀, C(O)OR₉, C(O)NH₂, C(O)NHR₁₀, C(O)N(R₁₀)₂, alkyl, haloalkyl, C₃-C₁₀ cycloalkyl containing 0-3 R₁₁, C₃-C₁₀ heterocyclyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl heteroaryl containing 0-3 R₁₂, or C₆-C₂₀ heteroaryl containing 0-3 R₁₂; or C₂-C₆ alkenyl, C₂-C₆ alkynyl, aryl, or heteroaryl optionally substituted with one or more halo, hydroxyl, OR₉, OR₁₀, acyloxy, nitro, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₁₀, sulfate, phosphate, cyano, carboxyl, C(O)R₉, C(O)R₁₀, C(O)OR₉, C(O)NH₂, C(O)NHR₉, C(O)N(R₉)₂, alkyl, haloalkyl, C₃-C₁₀ cycloalkyl containing 0-3 R₁₁, C₃-C₁₀ heterocycyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl containing 0-3 R₁₂, or C₆-C₂₀ heteroaryl containing 0-3 R₁₂;
R₁₇ is —H or C₁-C₆ alkyl;
R₃ is —H if R4 is —OH, or R₃ is —OH if R₄ is —H;
R₆ is —OH or forms a double bond with R₇;
R₇ is —H or forms a double bond with R₆;
R₈ is a saturated or unsaturated hydrocarbon having from about 5 to about 15 carbons;
each R₉ is independently a C₁-C₂₀ alkyl optionally substituted with halo, hydroxyl, alkoxy, amino, alkylamino, dialkylamino, sulfate, or phosphate;
each R₁₀ is independently an aryl optionally substituted with halo, haloalkyl, hydroxyl, alkoxy, nitro, amino, alkylamino, dialkylamino, sulfate, or phosphate;
each R₁₁ is independently halo, haloalkyl, hydroxyl, alkoxy, oxo, amino, alkylamino, dialkylamino, sulfate, or phosphate; and
each R₁₂ is independently halo, haloalkyl, hydroxyl, alkoxy, nitro, amino, alkylamino, dialkylamino, sulfate, or phosphate.

In some embodiments of formula II, R₁ is C(O) R₁₃ where R₁₃ is C₁-C₁₂ alkyl, R₂ is H, R₆ is OH or forms a double bond with R₇, and R₈ is a saturated or unsaturated hydrocarbon having from about 5 to about 15 carbons.

In some embodiments of formula II, X is O, R₁ is C(O) R₁₃ where R₁₃ is C₁-C₁₂ alkyl, R₂ is H, R₆ is OH or forms a double bond with R₇, and R₈ is a saturated or unsaturated hydrocarbon having from about 5 to about 15 carbons. Examples include but are not limited to

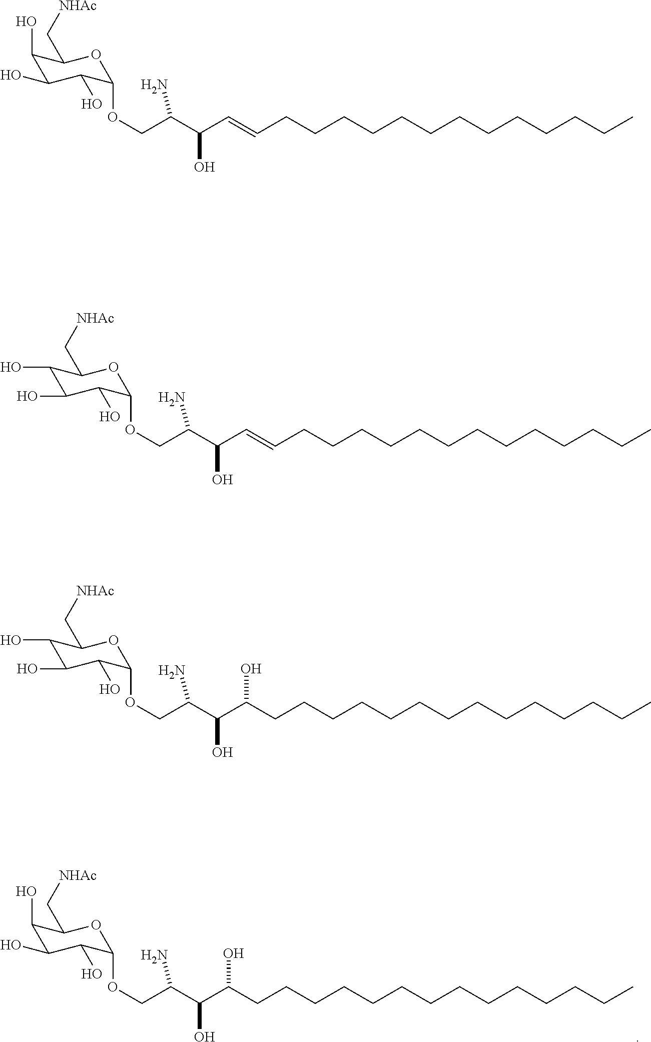 [SMILES: CCCCCCCCCCCCC/C=C/[C@@H](O)[C@@H](N)COC1OC(CNC(C)=O)C(O)C(O)C1O ; CCCCCCCCCCCCC/C=C/[C@@H](O)[C@@H](N)COC1OC(CNC(C)=O)C(O)C(O)C1O ; CCCCCCCCCCCCCC[C@@H](O)[C@@H](O)[C@@H](N)COC1OC(CNC(C)=O)C(O)C(O)C1O ; CCCCCCCCCCCCCC[C@@H](O)[C@@H](O)[C@@H](N)COC1OC(CNC(C)=O)C(O)C(O)C1O]

In another aspect, compounds of the disclosure are glycolipids represented by formula III:

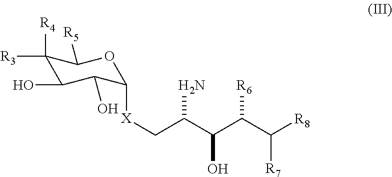

wherein:
X is O, S, or CH₂;
R₃ is —H if R₄ is —OH, or R₃ is —OH if R₄ is —H;
R₅ is —SR₁₅ or —OR₁₅;
   wherein R₁₅ is C₁-C₁₂ alkyl optionally substituted with halo, hydroxyl, oxo, nitro, OR₉, OR₁₀, acyloxy, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₉, NHSO₂R₁₀, sulfate, phosphate, cyano, carboxyl, C(O)R₉, C(O)R₁₀, C(O)OR₉, C(O)NH₂, C(O)NHR₉, C(O)N(R₉)₂, C₃-C₁₀ cycloalkyl containing 0-3 R₁₁, C₃-C₁₀ heterocycyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl containing 0-3 R₁₂, or heteroaryl containing 0-3 R₁₂; or C₃-C₁₀ cycloalky or C₅-C₁₀ cycloalkenyl optionally substituted with one or more halo hydroxyl, oxo, OR₉, OR₁₀, acyloxy, nitro, amino, NHR₉, N(R₉)₂, NHR₁₀, N(R₁₀)₂, aralkylamino, mercapto, thioalkoxy, S(O)R₉, S(O)R₁₀, SO₂R, SO₂R₁₀, NHSO₂R₉, NHSO₂R₁₀, sulfate, phosphate, cyano, carboxyl, C(O)R₉, C(O)R₁₀, C(O)OR₉, C(O)NH₂, C(O)NHR₁₀, C(O)N(R₁₀)₂, alkyl, haloalkyl, C₃-C₁₀ cycloalkyl containing 0-3 R₁₁, C₃-C₁₀ heterocyclyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl heteroaryl containing 0-3 R₁₂, or C₆-C₂₀ heteroaryl containing 0-3 R₁₂; or C₂-C₆ alkenyl, C₂-C₆ alkynyl, or aryl, optionally substituted with one or more halo, hydroxyl, OR₉, OR₁₀, acyloxy, nitro, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₁₀, sulfate, phosphate, cyano, carboxyl, C(O)R₉, C(O)R₁₀, C(O)OR₉, C(O)NH₂, C(O)NHR₉, C(O)N(R₉)₂, alkyl, haloalkyl, C₃-C₁₀ cycloalkyl containing 0-3 R₁₁, C₃-C₁₀ heterocycyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl containing 0-3 R₁₂, or C₆-C₂₀ heteroaryl containing 0-3 R₁₂;
R₆ is —OH or forms a double bond with R₇;
R₇ is —H or forms a double bond with R₆;
R₈ is a saturated or unsaturated hydrocarbon having from about 5 to about 15 carbons;
each R₉ is independently a C₁-C₂₀ alkyl optionally substituted with halo, hydroxyl, alkoxy, amino, alkylamino, dialkylamino, sulfate, or phosphate;
each R₁₀ is independently an aryl optionally substituted with halo, haloalkyl, hydroxyl, alkoxy, nitro, amino, alkylamino, dialkylamino, sulfate, or phosphate;
each R₁₁ is independently halo, haloalkyl, hydroxyl, alkoxy, oxo, amino, alkylamino, dialkylamino, sulfate, or phosphate; and
each R₁₂ is independently halo, haloalkyl, hydroxyl, alkoxy, nitro, amino, alkylamino, dialkylamino, sulfate, or phosphate.

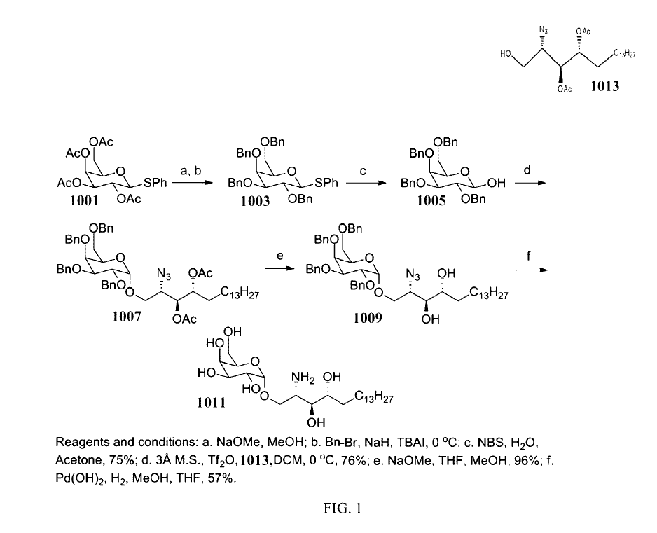

FIG. 1 shows a first scheme for synthesizing compounds according to the disclosure. In this embodiment, α-psychosine may be synthesized by starting from compound 1001. The acetates are removed with sodium methoxide, which leaves naked hydroxyls that are protected with benzyl bromide to give perbenzylated compound 1003. Transformation of the thio phenyl to a hydroxyl group at the anomeric position is then accomplished with n-bromosuccinimide with water and acetone as the solvent, resulting in compound 1005. Using donor 1005 and acceptor 1013 under coupling conditions disclosed in Garcia et al., 1997, J. Amer. Chem. Soc., 119: 7597-7598 results in compound 1007. In this embodiment, the anomeric effect biases towards the α-anomer product 1007. The acetyl groups were removed with sodium methoxide in methanol, resulting in compound 1009. One-pot removal of the benzyl groups and reduction of the azide is with palladium hydroxide, producing α-psychosine 1011.

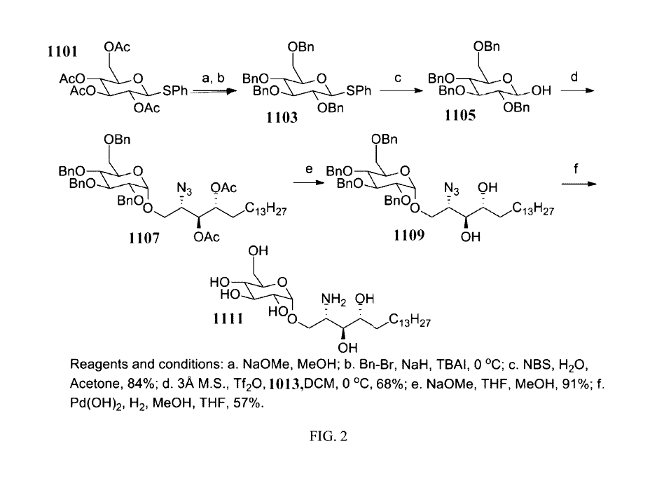

FIG. 2 shows a second scheme for synthesizing compounds according to the disclosure. In this embodiment, α-glucopsychosine may be synthesized by starting from compound 1101. The acetates are removed with sodium methoxide, which leaves naked hydroxyls that are protected with benzyl bromide to give perbenzylated compound 1103. Transformation of the thio phenyl to a hydroxyl group at the anomeric position is then accomplished with n-bromosuccinimide with water and acetone as the solvent, resulting in compound 1105. Using donor 1105 and acceptor 1013 under the coupling conditions disclosed in Garcia et al., 1997, J. Amer. Chem. Soc., 119: 7597-7598 results in compound 1107. In this embodiment, the anomeric effect biases towards the α-anomer product 1107. The acetyl groups were removed with sodium methoxide in methanol, resulting in compound 1109. One-pot removal of the benzyl groups and reduction of the azide is accomplished using palladium hydroxide, producing α-glucopsychosine 1111. Derivative compounds can be synthesized by modification of the schemes shown in FIGS. 1 and 2.

Preferably, compounds of the disclosure are capable of binding CD1d. The CD1d may be soluble, immobilized on a solid surface, or expressed on the surface of a cell, such as an antigen presenting cell or a cell transfected to express CD1d. Soluble CD1d, such as CD1d tetramers, are well known and commercially available. As used herein, “capable of binding a CD1d” means the ability of the compound to bind CD1d in a lipid binding assay. One example of such as assay is a competition assay of a charged glycolipid and an uncharged control and resolution of glycolipid-loaded CD1 molecules by isoelectric focusing (IEF) electrophoresis, as described for example in Cantu et al., 2003, J. Immunol., 170:4673-4682, the disclosure of which is incorporated herein by reference. As determined by IEF, binding of the compound to CD1d molecules can be quantified relative to binding of an uncharged glycolipid to CD1d molecules. Compound binding to CD1d can be titrated to saturation and quantified from the IEF gels to determine equilibrium binding constants. In an embodiment, a compound will be considered capable of binding a CD1d molecule if it displays a K_{D} less than 1 mM when determined using the assay in Cantu et al. cited above.

Other methods for assessing the ability of a compound to bind CD1d are known and include, e.g., gel filtration chromotagraphy, gel electrophoresis, surface plasmon resonance and ELISA. Binding may also be assessed by staining NKT cells with compounds of the disclosure complexed to CD1d tetramers, as described for example in Liu et al., 2006, J. Immun. Methods, 312: 34-39, incorporated herein by reference.

In embodiments, compounds of the disclosure are capable of activating an NKT cell. Activation of NKT cells can be assessed, e.g., as described below and in the examples.

### Compositions

Compositions comprising one or more compounds of formula I, formula II, formula III, or a combination thereof are provided. The compositions can include a physiologically acceptable vehicle. A “physiologically acceptable” vehicle is any vehicle that is suitable for in vivo administration (e.g., oral, transdermal or parenteral administration) or in vitro use, i.e., cell culture. Suitable physiologically acceptable vehicles for in vivo administration include water, buffered solutions and glucose solutions, among others. A suitable vehicle for cell culture is commercially available cell media. Additional components of the compositions may suitably include excipients such as stabilizers, preservatives, diluents, emulsifiers or lubricants, in addition to the physiologically acceptable vehicle and compound. In particular, suitable excipients include, but are not limited to, Tween 20, DMSO, sucrose, L-histadine, polysorbate 20 and serum.

Suitably, compositions comprising compounds of the disclosure may be formulated for in vivo use, i.e., therapeutic or prophylactic administration to a subject. The subject can be human. In some embodiments, the compositions are formulated for parenteral administration. A suitable dosage form for parenteral administration is an injectable. An injectable dosage form may be an isotonic solution or suspension and may be prepared using a suitable dispersion agent, wetting agent or suspension agent, as known in the art. In other embodiments, the compositions are formulated for oral administration. Suitable oral dosage forms include tablets, capsules, syrups, troches and wafers, among others. Oral dosage formulations suitably include lactose, starch, cellulose derivatives, magnesium stearate, stearic acid, glycols, and others. It will be appreciated that the compositions of the disclosure are not limited to any particular exemplified dosage form, but can be formulated in any manner described in the art, for example, in Remington: the Science and Practice of Pharmacy, 21^{st} ed., 2005, Lippincott Williams & Wilkins, Philadelphia, Pa.

In some embodiments, the compositions of the disclosure further include an antigen and are suitably formulated as a vaccine preparation. Antigens included in the compositions of the disclosure can be polypeptide or carbohydrate moieties, or combinations thereof, for example, glycoproteins. The antigen can be derived from an infectious agent (e.g., a pathogenic microorganism), a tumor, an endogenous molecule (e.g., a “self” molecule), or, for purposes of study, a nominal antigen, such as ovalbumin. A vaccine can be formulated using a variety of preparative methods known to those of skill in the art. See, for example, Remington: the Science and Practice of Pharmacy, 21^{st} ed., 2005, Lippincott Williams & Wilkins, Philadelphia, Pa.

In some embodiments, antigens for inclusion in compositions of the disclosure are suitably derived from attenuated or killed infectious agents. It will be understood that whole microorganisms or portions thereof (e.g., membrane ghosts; crude membrane preparations, lysates and other preparations of microorganisms) may suitably be included as an antigen. Suitable infectious agents from which an antigen may be derived include, but are not limited to, pathogenic viruses and microorganisms. In some contexts, suitable antigens are obtained or derived from a viral pathogen that is associated with human disease including, but not limited to, HIV/AIDS (Retroviridae, e.g., gp120 molecules for HIV-1 and HIV-2 isolates, HTLV-I, HTLV-11), influenza viruses (Orthomyxoviridae, e.g., types A, B and C), herpes (e.g., herpes simplex viruses, HSV-1 and HSV-2 glycoproteins gB, gD and gH), rotavirus infections (Reoviridae), respiratory infections (parainfluenza and respiratory syncytial viruses), Poliomyelitis (Picornaviridae, e.g., polioviruses, rhinoviruses), measles and mumps (Paramyxoviridae), Rubella (Togaviridae, e.g., rubella virus), hepatitis (e.g., hepatitis viruses types A, B, C, D, E and/or G), cytomegalovirus (e.g., gB and gH), gastroenteritis (Caliciviridae), Yellow and West Nile fever (Flaviviridae), Rabies (Rhabdoviridae), Korean hemorrhagic fever (Bunyaviridae), Venezuelan fever (Arenaviridae), warts (Papillomavirus), simian immunodeficiency virus, encephalitis virus, varicella zoster virus, Epstein-Barr virus, and other virus families, including Coronaviridae, Birnaviridae and Filoviridae.

Suitable bacterial and parasitic antigens can also be obtained or derived from known bacterial agents responsible for diseases including, but not limited to, diphtheria, pertussis, tetanus, tuberculosis, bacterial or fungal pneumonia, otitis media, gonorrhea, cholera, typhoid, meningitis, mononucleosis, plague, shigellosis or salmonellosis, Legionnaires' disease, Lyme disease, leprosy, malaria, hookworm, Onchocerciasis, Schistosomiasis, Trypanosomiasis, Leishmaniasis, giardiases, amoebiasis, filariasis, Borrelia, and trichinosis. Still further antigens can be obtained or derived from unconventional pathogens such as the causative agents of kuru, Creutzfeldt-Jakob disease (CJD), scrapie, transmissible mink encephalopathy, and chronic wasting diseases, or from proteinaceous infectious particles such as prions that are associated with mad cow disease.

Specific pathogens from which antigens can be derived include M. tuberculosis, Chlamydia, N. gonorrhoeae, Shigella, Salmonella, Vibrio cholerae, Treponema pallidum, Pseudomonas, Bordetella pertussis, Brucella, Francisella tularensis, Helicobacter pylori, Leptospira interrogans, Legionellapneumophila, Yersiniapestis, Streptococcus (types A and B), pneumococcus, meningococcus, Haemophilus influenza (type b), Toxoplasma gondii, Moraxella catarrhalis, donovanosis, and actinomycosis; fungal pathogens include candidiasis and aspergillosis; parasitic pathogens include Taenia, flukes, roundworms, amebiasis, giardiasis, Cryptosporidium, Schistosoma, Pneumocystis carinii, trichomoniasis and trichinosis. The present invention can also be used to provide a suitable immune response against numerous veterinary diseases, such as foot-and-mouth diseases, coronavirus, Pasteurella multocida, Helicobacter, Strongylus vulgaris, Actinobacillus pleuropneumonia, Bovine Viral Diarrhea Virus (BVDV), Klebsiella pneumoniae, E. coli, and Bordetella pertussis, parapertussis and brochiseptica.

In some embodiments, antigens for inclusion in compositions of the invention are suitably tumor-derived antigens or autologous or allogeneic whole tumor cells. Suitably, the tumor antigen is a tumor specific antigen (TSA) or a tumor associated antigen (TAA). Several tumor antigens and their expression patterns are known in the art and can be selected based on the tumor type to be treated. Non-limiting examples of tumor antigens include cdk4 (melanoma), β-catenin (melanoma), caspase-8 (squamous cell carcinoma), MAGE-1 and MAGE-3 (melanoma, breast, glioma), tyrosinase (melanoma), surface Ig idiotype (e.g., BCR) (lymphoma), Her-2/neu (breast, ovarian), MUC-1 (breast, pancreatic) and HPV E6 and E7 (cervical carcinoma). Additional suitable tumor antigens include prostate specific antigen (PSA), sialyl Tn (STn), heat shock proteins and associated tumor peptides (e.g., gp96), ganglioside molecules (e.g., GM2, GD2, and GD3), Carcinoembryonic antigen (CEA) and MART-1.

### Methods of Activating NKT Cells

Methods of activating NKT cells with a compound or composition of the disclosure are provided. “Stimulating an NKT cell” and “activating an NKT cell” are used interchangeably herein to refer to inducing an observable effect in an NKT cell that is consistent with a cellular response to engagement of the TCR of the NKT cell with an antigen presented in the context of CD1d molecule. Observable effects of activation of NKT cells include secretion of cytokines, clonal proliferation and upregulation of expression of cell surface markers, for example, CD69 molecules, IL-12 receptors and/or CD40L molecules. To activate an NKT cell in accordance with the present methods, the NKT cell is contacted with a compound or composition of the disclosure in the presence of CD1d. Suitably, a compound of the disclosure stimulates an NKT cell when the compound is complexed with, or bound to, a CD1d molecule. Activation of the NKT cell results from contacting the TCR of the NKT cell with the complex, thereby eliciting an observable response, such as, e.g., altered cytokine expression. A “T cell receptor of an NKT cell,” as the term is used herein, refers to the conserved, semi-invariant TCR of NKT cells comprising e.g., Vα14-Jα18N/Vβ11 in humans and Vβ14-Jα18N/Vβ8 in mice.

As used herein, “contacting an NKT cell” refers to the in vitro addition of a compound of the invention to NKT cells in culture, optionally in the presence of immobilized, soluble, or insoluble CD1d or cells, such as antigen presenting cells (APCs), expressing CD1d molecules, or to the in vivo administration of a compound or composition of the disclosure to a subject. In embodiments, the compound presented to the TCR of the NKT cell by CD1d molecules on the surface of an antigen presenting cell (APC), such as a dendritic cell (DC) or macrophage. Alternatively, CD1d molecules may be plated and the NKT cells and a compound of the invention can be added to the CD1d molecules in vitro.

Examples of cytokines that may be secreted by NKT cells activated in accordance with the disclosure include, but are not limited to, IL-10, IL-4, and IL-12, IL-13, GM-CSF, IFN-γ IL-2, IL-1, IL-6, IL-8, TNF-α, and TGF-8. It is appreciated that combinations of any of the above-noted cytokines may be secreted by NKT cells upon activation and used to detect NKT cell activation. Methods for detecting and measuring levels of secreted cytokines are well known in the art. As will be appreciated, assessing NKT cell activation is suitably accomplished by measuring cytokine expression by the NKT cell relative to a suitable control. One example of a T cell activation assay for detecting NKT cell activation via IL-2 is provided in the examples.

NKT cell proliferation may also be induced by contacting NKT cells with one or more compounds of the disclosure. Proliferation is suitably measured in vitro by standard methods, e.g. ³H-thymidine or BrdU incorporation assays.

Upregulation of cell surface markers is also suitably observed upon activation of NKT cells. For example, CD69, CD25, CD40L and IL-12 receptors are upregulated upon activation of NKT cells. Immunologic methods, such as FACS, can be used to detect upregulation of cell surface markers, as well as other methods commonly employed in the art. Downstream effects of NKT cell activation, such as induction of DC maturation, are also observable, e.g., by measuring upregulation of CD80 and/or CD86 on DCs.

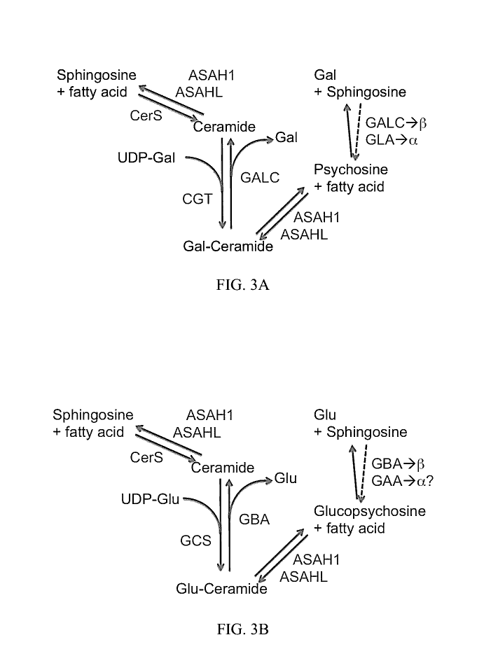

As shown in FIGS. 3A and 3B, production of α-glucosyl and galactosylceramides were found to be controlled by catabolic enzymes and the availability of α-glycosylceramides in antigen presenting cells, such as dendritic cells, was directly controlled by catabolic enzymes. In FIGS. 3A and 3B, ASAH1, ASAHL, CerS, CGT, GALC, and GLA represent enzymes, and the other components represent products.

The activation of NKT cells can be induced or enhanced by contacting antigen presenting cells with an inhibitor of one or more enzymes and/or transfer proteins in the lysosome, including but not limited to catabolic enzymes, including but not limited to α-glycosidases, such as α-glucosidase and α-galactosidase, and ceramidase as shown in FIGS. 3A and 3B, and lipid transfer proteins, including but not limited to saposin B and GM2A. The inhibitors can be a drug, small molecule, peptide, or antibody, such as an intracellular antibody. A small molecule is generally a low molecular weight (e.g., <900 Daltons) organic compound. Useful inhibitors include but are not limited to 1-Deoxynojirimycin, N-[(1R,2R)-2-hydroxy-1-(hydroxymethyl)-2-(4-nitrophenyl)ethyl]-tetradecanamide (D-NMAPPD), E)-3-(3-(4-methoxyphenyl)acryloyl)-4-phenylquinolin-2(1H)-one (Ceranib-2), 1-Deoxygalactononojirimycin. 1-(2-Biphenyl-4-yl)ethyl-carbonyl pyrrolidine (NAAA inhibitor), and carmofur (1-Hexylcarbamoyl-5-fluorouracil).

The activation of NKT cells can be reduced or inhibited by contacting antigen presenting cells with an agent that interferes with lysosome acidification and/or increases the pH in the lysosome to reduce or inhibit production of α-glycosylceramides. Examples of suitable agents include but are not limited to chloroquine and derivatives of chloroquine, including but not limited to chloroquine diphosphate, chloroquine phosphate, chloroquine sulfate, chloroquine dihydrochloride, dichloroquine primaquine, amodiaquine, piperaquine, and mefloquine.

In vivo and ex vivo activation of NKT cells is specifically contemplated in addition to in vitro activation. Presentation of compounds of the disclosure to NKT cells in the context of CD1d molecules results in NKT cell activation and dendritic cell maturation. Consequently, these compounds stimulate immune responses against nominal antigens as well as infectious agents and neoplastic malignancies, including solid and hematologic tumors. Both cellular and humoral immunity may be stimulated by administering NKT cell agonist compounds, as described herein.

Methods of stimulating an NKT cell in vivo, i.e., in a subject, include administering a NKT cell agonist compound to the subject and/or an inhibitor of the catabolic enzymes regulating expression and/or availability of α-glycosylceramides in antigen presenting cells. In an embodiment, administration to a subject in accordance with methods of the disclosure can include first formulating the NKT cell agonist compound or inhibitor of the catabolic enzymes with a physiologically acceptable vehicle and/or excipient to provide desired dosages, stability, etc. Suitable formulations for vaccine preparations and therapeutic compounds are known in the art.

Methods of stimulating an NKT cell ex vivo may include use of adoptive transfer methods based on administering cells that have been contacted with NKT cell agonist compounds ex vivo to stimulate NKT cells in a subject. In some embodiments, the cells may be NKT cells that are stimulated ex vivo and injected into a subject. In some embodiments, the cells may be APCs that have been contacted ex vivo with compounds of the disclosure to allow loading of the surface-expressed CD1d molecules with the compound for presentation to NKT cells. In other embodiments, the cells may be APCs that have been contacted ex vivo with one or more inhibitors of enzymes in the catabolic pathway to induce and/or enhance expression and availability of α-glycosylceramides by the APCs. The ex vivo stimulated NKT cells and/or treated APCs can then be administered, e.g., by injection into the subject.

### Methods of Stimulating an Immune Response

Methods of stimulating an immune response in a subject with a compound or composition of the disclosure are also provided. A “subject” is a vertebrate, suitably a mammal, more suitably a human. As will be appreciated, for purposes of study, the subject is suitably an animal model, e.g., a mouse. “Stimulating an immune response” includes, but is not limited to, inducing a therapeutic or prophylactic effect that is mediated by the immune system of the subject. More specifically, stimulating an immune response in the context of the disclosure refers to eliciting an NKT cell response in a subject by administering an effective amount of a compound or composition of the disclosure to the subject, thereby inducing downstream effects such as production of antibodies, antibody heavy chain class switching, maturation of APCs, and stimulation of cytolytic T cells, T helper cells and both T and B memory cells. Alternatively, stimulation of an immune response in a subject can be accomplished by administering to the subject one or more inhibitors of enzymes in the catabolic pathway of the disclosure to induce and/or enhance expression and availability of α-glycosylceramides by the APCs. Alternatively, stimulation of an immune response in a subject may be accomplished by administering to the subject a population of NKT cells that have been activated as described herein. Alternatively, stimulation of an immune response in a subject may be accomplished by administering to the subject a population of CD1d+ antigen presenting cells that have been contacted with a compound of the disclosure. Alternatively, stimulation of an immune response in a subject may be accomplished by administering to the subject a population of APCs that have been contacted with one or more inhibitors of enzymes in the catabolic pathway of the disclosure to induce or enhance expression and availability of α-glycosylceramides by the APCs. Any combination of the above methods of stimulating an immune response may be suitable.

In some embodiments, the immune response stimulated according to the disclosure is an antimicrobial immune response. Such an immune response suitably promotes clearance of an infectious agent or permits immune control of the agent such that disease symptoms are reduced or resolved, e.g., a persistent or latent infection.

In other embodiments, the enhanced immune response is an anticancer or antitumor immune response. Such an immune response suitably promotes tumor rejection, reduces tumor volume, reduces tumor burden, prevents metastasis, and/or prevents recurrence of the tumor. The tumor may be any solid or hematologic tumor, including but not limited to leukemia, lymphoma, AIDS-related cancers, cancers of the bone, brain, breast, gastrointestinal system, endocrine system, eye, genitourinary tract, germ cells, reproductive organs, head and neck, musculoskeletal system, skin, nervous system or respiratory system. As is appreciated in the art, a cancer-specific immune response may be monitored by several methods, including: 1) measuring cytotoxicity of effector cells, using, e.g., a chromium release assay; 2) measuring cytokine secretion by effector cells; 3) evaluating T cell receptor (TCR) specificities, e.g., by using MHC-peptide multimers; 4) measuring the clonal composition of the T cell response; and/or 5) measuring T cell degranulation.

An enhanced immune response is also suitably assessed by the assays such as, e.g. activation of NKT cells, inducing cytokine production, inducing maturation of APCs, enhancing cytolytic and helper T cell functions, enhancing CD8+ and CD4+ T cell recruitment, enhancing antibody production, inducing antibody class switching, and breaking tolerance.

In some embodiments, stimulating an immune response in a subject in accordance with the disclosure can be accomplished by administering to the subject a composition including a compound of the invention. In some embodiments, the composition is administered to the subject with an antigen. The compound and the antigen may or may not induce a detectably enhanced immune response when administered to a subject independently. In other embodiments, stimulating an immune response in a subject in accordance with the disclosure can be accomplished by administering to the subject one or more inhibitors of enzymes in the catabolic pathway of the disclosure to induce and/or enhance expression and availability of α-glycosylceramides by the APCs. In some embodiments, the one or more inhibitors are administered to the subject with an antigen. The one or more inhibitors and the antigen may or may not induce a detectably enhanced immune response when administered to a subject independently.

The antigen and the compound and/or inhibitor of an enzyme in the catabolic pathway of the disclosure can be co-administered to stimulate an immune response in a subject. The term “co-administration” refers to any administration protocol in which a compound or inhibitor of the disclosure and an antigen are administered to a subject. The antigen and the compound or inhibitor can be in the same dosage formulations or separate formulations. Where the antigen and compound or inhibitor are in separate dosage formulations, they can be administered concurrently, simultaneously or sequentially (i.e., administration of one may directly follow administration of the other or they may be given episodically, i.e., one can be given at one time followed by the other at a later time, e.g., within a week), as long as they are given in a manner sufficient to allow both to achieve therapeutically or prophylactically effective amounts in the subject. The antigen and the compound or inhibitor can also be administered by different routes, e.g., one may be administered intravenously while the second is administered intramuscularly, intravenously or orally.

In some embodiments, the compound or inhibitor is suitably added to a vaccine composition or is co-administered with a vaccine composition. Addition of a compound of the disclosure to a vaccine composition or co-administration with a vaccine composition may be particularly suitable in cases where the antigen has a low rate of efficacy as a vaccine and/or must be administered in an amount or at a dose greater than what might be considered ideal due to side effects, cost and/or availability of the antigen, etc. Examples of such vaccines may include, but are not limited to human papillomavirus vaccines, acute otitis media vaccine (PREVNAR®), influenza vaccines, cholera vaccines, and the telomerase cancer vaccine.

Administration to a subject can be carried out by any suitable method, including intraperitoneal, intravenous, intramuscular, subcutaneous, transcutaneous, oral, nasopharyngeal, or transmucosal absorption, among others. Suitably, a compound of the disclosure is administered in an amount effective to activate an NKT cell or cells such that a prophylactic or therapeutic effect is achieved in the subject, e.g., an antitumor immune response or antimicrobial immune response.

Administration to a subject also includes use of adoptive transfer methods based on administering cells that have been contacted with a compound of the disclosure ex vivo to stimulate or enhance an immune response in a subject. In some embodiments, the cells may be NKT cells that are activated ex vivo and injected into a subject to provide or enhance an immune response to, e.g., cancerous cells or infectious agents. In some embodiments, the cells may be APCs that have been contacted with a compound of the disclosure ex vivo to allow complexing with the CD1d molecules expressed by the APC. In other embodiments, the cells may be APCs that have been contacted ex vivo with one or more inhibitors of enzymes in the catabolic pathway to induce and/or enhance expression and availability of α-glycosylceramides by the APCs. Antigen presenting cells can then be administered, e.g., by injection into the subject, to provide a suitable immune response. This method of administration allows for stimulation of the immune response with minimal exposure of the subject or the subject's cells to the compounds.

Administration of compounds of the disclosure or an inhibitor of an enzyme in the catabolic pathway as described herein to a subject in accordance with the disclosure can exhibit beneficial effects in a dose-dependent manner. Thus, within broad limits, administration of larger quantities of the compounds or an inhibitor is expected to activate greater numbers of NKT cells or activate NKT cells to a greater degree than does administration of a smaller amount. Moreover, efficacy is also contemplated at dosages below the level at which toxicity is seen.

It will be appreciated that the specific dosage administered in any given case will be adjusted in accordance with the compound or compounds being administered, the disease to be treated or prevented, the condition of the subject, and other relevant medical factors that may modify the activity of the compound or the response of the subject, as is well known by those skilled in the art. For example, the specific dose for a particular patient depends on age, body weight, general state of health, diet, the timing and mode of administration, the rate of excretion, medicaments used in combination, and the severity of the particular disorder to which the therapy is applied. Dosages for a given patient can be determined using conventional considerations, e.g., by customary comparison of the differential activities of the compound of the disclosure and of a reference agent such as αGalCer, such as by means of an appropriate conventional pharmacological or prophylactic protocol.

The maximal dosage for a subject is the highest dosage that does not cause undesirable or intolerable side effects. The number of variables in regard to an individual prophylactic or treatment regimen is large, and a considerable range of doses is expected. It is anticipated that dosages of compounds of the disclosure will prevent or reduce symptoms at least 50% compared to pre-treatment symptoms. It is specifically contemplated that vaccine preparations and compositions of the invention may palliate or alleviate symptoms of the disease without providing a cure, or, in some embodiments, can be used to cure or prevent the disease or disorder.

Suitable effective dosage amounts for administering the compounds of the disclosure may be determined by those of skill in the art, but typically range from about 1 microgram to about 10,000 micrograms per kilogram of body weight weekly, although they are typically about 1,000 micrograms or less per kilogram of body weight weekly. In some embodiments, the effective dosage amount ranges from about 10 to about 5,000 micrograms per kilogram of body weight weekly. In another embodiment, the effective dosage amount ranges from about 50 to about 1,000 micrograms per kilogram of body weight weekly. In another embodiment, the effective dosage amount ranges from about 75 to about 500 micrograms per kilogram of body weight weekly. The effective dosage amounts described herein refer to total amounts administered, that is, if more than one compound is administered, the effective dosage amounts correspond to the total amount administered. The compound or inhibitor can be administered as a single weekly dose or as divided doses.

In some embodiments, a tumor antigen and the compound or an inhibitor of the disclosure are co-administered to a subject to induce an anti-tumor immune response in the subject. Suitably, co-administration of the antigen with the compound or inhibitor of the disclosure enhances the anti-tumor response and results in inhibition of tumor growth, reduction in tumor burden and treatment of cancer, as described herein.

In some embodiments, compounds of formula I, formula II, or formula III are cytotoxic and useful in chemotherapy for the treatment of cancer. In some embodiments, compounds of formula I, formula IL, or formula III are capable of inducing apoptosis in cells, such as tumor cells or cancer cells. In embodiments, the compound can be formulated in a composition as described herein and administered to a subject to treat cancer. Such compounds can be administered with a tumor antigen to provide a dual mode for treating cancer in which the compounds are both cytotoxic to cancer cells and capable of inducing an anti-tumor response in combination with the tumor antigen.

### Methods of Modulating NKT Cell Activation

Methods of modulating activation of NKT cells are also provided. “Modulating” as used herein can refer to stimulating and/or enhancing NKT cell activation in a subject if the subject would benefit from such activation or increase in NKT cell activation. Methods of stimulating NKT cells and methods of treating a disease or disorder in which the subject would benefit from NKT cell activation or an increase in NKT cell activation are discussed above.

“Modulating” as used herein can also refer to reducing and/or inhibiting activation of NKT cells in a subject if the subject would benefit from such a reduction and/or inhibition of NKT cell activation. Such methods can be used to treat a subject having a disease or disorder in which activation of NKT cells contributes to or is causative of the disease or disorder. Examples of such diseases and disorders include but are not limited to autoimmune disorders, including but not limited to type I diabetes, rheumatoid arthritis, systemic lupus erythematosus, primary biliary cirrhosis, hepatitis, and multiple sclerosis, and allergy disorders including but not limited to asthma, atopic dermatitis, eczema, and allergic rhinitis.

As shown in the examples, activation of NKT cells can be reduced by inhibiting the interaction of α-glycosylceramides with NKT cells. Examples of suitable NKT cell activation antagonists include but are not limited to antibodies that bind α-glycosylceramides, antibodies that bind the complex formed by α-glycosylceramide and CDld, agents that interfere with lysosomal acidification and/or increase the pH in the lysosome to reduce or inhibit production of α-glycosylceramides, agents that inhibit or interfere with saposins in the lysosome, and variant α-glycosylceramides that bind to CD1d but exhibit reduced NKT cell stimulatory activity in combination with CD1d.

Examples of agents capable of interfering with lysosomal acidification and/or increasing the pH in the lysosome include but are not limited to, chloroquine and derivatives of chloroquine, including but not limited to, chloroquine diphosphate, chloroquine phosphate, chloroquine sulfate, chloroquine dihydrochloride, dichloroquine primaquine, amodiaquine, piperaquine, and mefloquine.

Examples of antibodies include but are not limited to antibodies L317 and L363, which are further described in the examples. In some embodiments the antibody binds to the complex formed by α-glycosylceramide and CD1d and sterically hinders the binding of the loaded CD1d with NKT cells. Antibodies that bind α-glycosylceramides can be made according to known methods, including methods of obtaining polyclonal antibodies, methods of obtaining monoclonal antibodies, methods of phage display, methods to generate human or humanized antibodies, and methods using a transgenic animal or plant engineered to produce human antibodies or humanized antibodies. Polyclonal antibodies can be produced by various procedures well known in the art. For example, an α-glycosylceramide or CD1d complexed α-glycosylceramide can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981).

Phage display libraries of partially or fully synthetic antibodies are available and can be screened for an antibody or fragment thereof that can bind to α-glycosylceramide, such as α-glucosylceramide or α-galactosylceramide. Phage display libraries of human antibodies are also available. In embodiments, antibodies specifically bind to α-glucosylceramide and/or α-galactosylceramide and do not cross react with nonspecific components such as serum albumins or other unrelated antigens. Once identified, the amino acid sequence or polynucleotide sequence coding for the antibody can be isolated and/or determined.

Antibodies can be humanized, primatized, deimmunized, synthetic or chimeric antibodies. These types of antibodies are derived from a non-human antibody, typically a murine or primate antibody, that retains or substantially retains the antigen-binding properties of the parent antibody, but which is less immunogenic in humans. This may be achieved by various methods, for example, but not limited to, (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; (c) transplanting the entire non-human variable domains, but “cloaking” them with a human-like section by replacement of surface residues; or (d) use of genetically modified mice wherein the mouse engineered to express human repertoire, for example, human immunoglobulin heavy and light chain variable domains. Such methods are disclosed, for example, in Morrison et al., Proc. Natl. Acad. Sci. 81:6851-6855 (1984); Morrison et al., Adv. Immunol. 44:65-92 (1988); Verhoeyen et al., Science 239:1534-1536 (1988); Padlan, Molec. Immun. 28:489-498 (1991); Padlan, Molec. Immun. 31:169-217 (1994), Peterson, ILAR Journal 46(3): 314-319 (2005), Lonberg, Nat. Biotechnol. 23(9): 1119-1125 (2005) and U.S. Pat. Nos. 5,585,089, 5,693,761, 5,693,762, 6,190,370, and US2012/0021409.

Antibody as used herein includes antigen binding fragments and includes all or a portion of polyclonal antibodies, a monoclonal antibody, a human antibody, a humanized antibody, a synthetic antibody, a chimeric antibody, a bispecific antibody, a minibody, and a linear antibody. Antibody fragments comprise a portion of an intact antibody, preferably the antigen binding or variable region of the intact antibody and can readily be prepared using conventional methods. Examples of suitable antibody fragments for use in the methods of the disclosure include Fab, Fab′, F(ab′)₂, and Fv fragments; diabodies; linear antibodies; single-chain antibody molecules; and multispecific antibodies formed from antibody fragments. Antibodies that bind α-glucosylceramides and/or α-galactosylceramides can be screened for NKT cell modulating activity (e.g., agonist activity or antagonist activity) using CD1d binding assays and NKT cell activation assays as described herein. Identified agonist or antagonist antibodies can be formulated and administered to a subject as described herein for the compounds of the disclosure.

Variants of α-glucosylceramides and α-galactosylceramides can be made as described for example in U.S. Pat. No. 7,645,873, and U.S. Pat. No. 8,227,581, which are hereby incorporated by reference. Structural changes can be made to the exposed carbohydrate of α-glucosylceramide and/or α-galactosylceramide to generate variant compounds that affect NKT cell stimulation activity. The replacement of the parent sugar's C₆″-hydroyl by a more reactive amino group allows for the efficient synthesis of C₆″-amino-C₆″-deoxyglycosylceramides. The derivitization handle situated at C₆″ allows the exposed carbohydrate group of α-glucosylceramide and α-galactosylceramide to be modified without significantly altering CD1d binding affinity because the C₆″-amino substituents are sufficiently distanced from the lipid portion of the molecule which interacts with the deep hydrophobic pocket of CD1d. Additional modifications can be made to the ceramide head group, lipid side chain, and/or sphingosine side chain to modulate NKT cell stimulatory activity. The variant α-glycosylceramides can be screened for NKT cell modulating activity (e.g., agonist activity or antagonist activity) using CD1d binding assays and NKT cell activation assays as described herein. Identified agonists or antagonists of NKT cell activation can be formulated and administered to a subject as described herein for the compounds of the disclosure.

### Methods of Identifying NKT Cell Agonists

In another aspect, methods of screening and identifying NKT cell agonists are disclosed. As shown in FIGS. 3A and 3B, production of α-glucosyl and galactosylceramides were found to be controlled by catabolic enzymes and the availability of α-glycosylceramides in antigen presenting cells, such as dendritic cells, was directly controlled by catabolic enzymes. The activation of NKT cells can be induced or enhanced by contacting antigen presenting cells with an inhibitor of one or more catabolic enzymes in the lysosome including but not limited to α-glycosidases, such as α-glucosidase and α-galactosidase, and ceramidase as shown in FIGS. 3A and 3B.

In an embodiment, antigen presenting cells are treated with a candidate inhibitor of ceramidase or a α-glycosidase, such as α-glucosidase or α-galactosidase. NKT cells are then contacted with the treated antigen presenting cells and the activation of the contacted NKT cells is determined using a T cell activation assay as described herein. One example of a T cell activation assay for detecting NKT cell activation via IL-2 is provided in the examples. Preferably the antigen presenting cells are CD1d+. In an embodiment, the antigen presenting cells are dendritic cells or thymocytes. In an embodiment, NKT cell activation is determined by comparing the contacted NKT cells to control NKT cells contacted with antigen presenting cells without the candidate inhibitor. An increase in NKT cell stimulation relative to the control NKT cells indicates that the candidate inhibitor is an NKT cell agonist. Identified NKT cell agonists can be further characterized and evaluated for use in stimulating NKT cell activation or an immune response in a subject as described herein.

### EXAMPLES

The following examples are illustrative and provided to assist in a further understanding of the disclosure. Other embodiments are within the scope of the present disclosure. The particular materials and conditions employed are intended to be further illustrative of the disclosure and are not limiting upon the reasonable scope thereof.

### Example 1

### Experimental Methods

The following materials and methods were used in the experiments described in Examples 2-6.

Chemicals and Inhibitors.

Lipopolysaccharide from Salmonella Abortus was obtained from Sigma. Recombinant IL-4, TNF, GM-CSF were obtained from InVitrogen. 1-Deoxynojirimycin, N-[(1R,2R)-2-hydroxy-1-(hydroxymethyl)-2-(4-nitrophenyl)ethyl]-tetradecanamide (D-NMAPPD), E)-3-(3-(4-methoxyphenyl)acryloyl)-4-phenylquinolin-2(1H)-one (Ceranib-2) were obtained from Cayman Chemical (Ann Arbor, Mich.). 1-Deoxygalactononojirimycin, 1-(2-Biphenyl-4-yl)ethyl-carbonyl pyrrolidine (NAAA inhibitor) was synthesized according to Li et al., 2012, PLoS One, 7:e43023. Carmofur (1-Hexylcarbamoyl-5-fluorouracil) was obtained from Sigma-Aldrich (St. Louis, Mo.). Synthetic commercial glucosylceramides and galactosylceramides were obtained from Avanti Polar Lipids (Alabaster, Ala.) and Matreya (Pleasant Gap, Pa.).

Cells and Cell Lines, DC Maturation.

DN32.D3 and TBA.7 cells have been described extensively and are commonly used as representative of type 1 semi-invariant Vα14 NKT cells for the former, and type 2 non-Vα14 NKT cell for the latter. DC3.2 cells are a dendritic cell line expressing CD1d and susceptible to differentiation induced by TLR ligands and cytokine such as LPS and TNF. Maturation of DC3.2 was carried over periods of 16-24 h.

Antibodies.

In most examples, anti-MHC class II antibodies MKD6 (anti-I-A^{d}, IgG2a) and 14.4.4s (anti-I-E^{k}, IgG2a) were used as a control. All antibodies were produced in serum-free Ultradoma media (Lonza) in individual bioreactors. Purification was carried out on HiTrap protein A or G columns (GE Healthcare, Pittsburgh, Pa.).

T Cell Activation Assay.

T cell hybridoma cells were cultured in RPMI supplemented with 10% FCS, 2 mM L-glutamine, 20 mM HEPES, and non-essential amino acids. Antigen presentation assays were carried out using 5-20×10³ DC 3.2 cells or 1×10⁵ splenocytes and 4×10⁴ T cells per well in 96 well tissue culture plates in triplicates. Cell culture supernatants were collected 24 h later for determination of IL-2 concentrations using an IL-2-dependent NK cell line reporter system.

Surface Plasmon Resonance (SPR).

A Biacore T200 instrument (GE Healthcare, Pittsburgh, Pa.) was used for SPR measurements. Measurements were performed using single cycle protocols to avoid repeated use of regeneration buffer on the immobilized ligands. Immobilization of target antibodies was carried out using classical amine coupling chemistry. 250 to 1,000 RU of antibody was immobilized in each flow cell. All mCD1-lipid complexes were purified after loading to ensure maximal homogeneity and avoid the presence of small amounts of aggregated material. Concentrations ranging from 1 to 10 mM were used for each CD1-lipid complex. Flow cell one was used as a negative control and used for subtraction from experimental flow cells. Global analysis of subtracted sensorgrams was carried out using the T200 analysis software.

Thin Layer Chromatography (TLC) and TLC-Blot.

TLC analysis was carried out using plates (EMD Bioscience, Billerica, Mass.). Running solutions were Chloroform/Methanol/25% Ammonium Hydroxide 90:20:0.5 for glycosylceramide and Chloroform/Methanol/CaCl2 60:40:9 for lysosphingolipids. Visualization was done using cerium-ammonium-molybdate stain (CAM) and heating to 100° C. Immunoblotting was performed using plates with aluminum support. Blocking solution was 3% non-fat dry milk in phosphate buffer saline (PBS) pH 7.4. After 2 h incubation with blocking buffer, plates were incubated overnight with antibodies diluted in blocking solution with gentle agitation. After extensive wash in PBS, binding was revealed using a IRDye 800CW anti-rabbit labeled antibody (Licor, Lincoln, Nebr.) on a LiCor imager. Purified anti-glucosylceramide rabbit serum was obtained from Glycobiotech.

Immunoprecipitation for Mass Spectrometry Analysis.

2×10⁹ cells were harvested, washed 3 times in PBS and incubated with 50 g of antibody for 2 h at room temperature on a rotating wheel, before being pelleted and lysed in 100 mM Tris pH 7.5, 150 mM NaCl, 0.1% Rapigest (Waters, Milford, Mass.). Cell debris were removed by centrifugation at 15,000 rpm for 30 min at 4° C. and 10 μg of antibody were added. After 2 h, antibody was recovered using protein-A Sepharose beads (GE Healthcare, Pittsburgh, Pa.). After extensive washes in phosphate buffer saline, lipids were directly extracted with a chloroform/methanol 2:1 mix by 2 min vortexing followed by centrifugation. Extraction was performed twice. Samples were directly used for mass spectrometry analysis.

LC and Mass Spectrometry.

An Agilent (Santa Clara, USA) 1200 UPLC system was coupled to a 6490 triple quadrupole mass spectrometer for use in C24:1 monoglycosylceramide determination using multiple reaction monitoring for enhanced sensitivity and selectivity. Samples were loaded onto an Agilent 300SB C8 2.1×100 mm column and separated using the following gradient: T=0 min 80% A to T=12 min 5% A. Stop time was at 17 min, with a 4.5 min re-equilibration time between runs. Mobile phase A consisted of 95:5 H₂O:MeOH, and B of 65:30:5 IPA:MeOH:H₂O. Flow rate=200 μl/min and 5 μl of each sample and standard was injected.

The 6490 triple quad mass spectrometer was equipped with a jet stream source with the following settings: gas temp=200° C., gas flow=14 L/min, nebulizer pressure=25 psi, sheath gas temp=350° C., sheath gas flow=10 L/min. Capillary voltage was maintained at 3500V, and nozzle voltage was set to 2000V. Two transitions were monitored to increase confidence: m/z 810.7->792.6 with collision energy set to 9V and 810.7->264.2 with collision energy at 41V. Fragmentor voltage was kept constant at 380V. Data was collected in positive ion mode.

Expression and Purification of Recombinant Molecules.

CD1d: Murine CD1d was produced as previously reported. Molecules are produced in S2 Drosophila melanogaster S2 cells and purified by successive NiNTA affinity and anion exchange chromatographies. Lipids are loaded onto “empty CD1d” molecules at pH 5.0 in 0.1M Malonate buffer, for 4 h at 37° C. in the presence of an equimolar amount of saposin B and a 20-fold excess of lipid. After isoelectric focusing gel to control loading, CD1-lipid complexes are purified by either gel filtration or anion exchange chromatography to ensure homogeneity and separation from free lipids and unloaded CD1d molecules.

GBA: Full length murine cDNA (1-1546) was modified to add a C-terminal histidine tag by PCR and cloned into a fly expression vector.

GLA: Full length murine cDNA (1-1260) was modified to add a C-terminal histidine tag by PCR and cloned into a fly expression vector.

Both GBA and GLA were expressed in serum-free media and purified by a succession of Ni-NTA and ion exchange chromatography. Enzymatic activity of the purified recombinant proteins was evaluated on synthetic commercial glucosylceramide (Avanti Polar Lipids, Alabaster, Ala.) and synthetic commercial globotriaosylceramide (Matreya, Pleasant Gap, Pa.) followed by TLC analysis. Both enzymes were highly active.

Flow Cytometry.

NKT cells were quantified using CD1d tetramers, empty or loaded with PBS-57. Tetramer and antibody staining was performed on single cell suspensions prepared from adult thymi and FTOC lobes. Organs were collected in cold flow buffer (FB; PBS containing 2% FCS/2 mM EDTA) and were passed through a 70 μm cell strainer to obtain a single cell solution. Samples were depleted of erythrocytes using 0.165M NH4Cl in water. Samples were washed twice and treated with Fc Block (BD Biosciences, San Jose, Calif.) and 0.5 mg/ml avidin (Sigma-Aldrich, St. Louis, Mo.) in FB at room temperature for 10 min. Cells were then washed with FB and stained with CD d/Empty or CD1d/PBS-57 tetramers at room temperature for 30 min. Anti-CD3ε and anti-B220 (BD Biosciences-Pharmingen, San Diego, Calif.) were directly added, and staining continued for another 20 min. Samples were washed twice in FB and propidium iodide was added for dead cell exclusion. Samples were acquired on a MACSQuant analyzer using MACSQuantify software (both Miltenyi Biotec, San Diego, Calif.) and analyzed with FlowJo software (Tree Star Inc., Ashland, Oreg.).

Fetal Thymic Organ Culture.

Embryonic day 14.5 fetal thymic lobes were harvested from timed pregnant C57BL/6J mice and cultured on nitrocellulose filters (Whatman) placed on a sponge (Gelfoam size 4; Upjohn Pharmacia, Peapack, N.J.). Lobes were cultured for 18 days in 0.5 mL DMEM (containing 10% FCS, 2 mM L-glutamine, 20 mM HEPES, non-essential amino acids and antibiotics) per well on 48-well tissue culture plates. Antibodies were added to the media throughout the culture period at a concentration of 60 μg/mL. Media was changed every 3 days. Cells were harvested by mechanical disruption of the thymic lobes, passaged through a 70 μm cell strainer and stained for flow cytometry.

### Example 2

### An Anti-CD1d-aGalCer Antibody Blocks Autoreactivity of CD1 Expressing Cells Towards NKT Cells

NKT cells have a memory phenotype and hallmarks of “pre-activation” when analyzed ex vivo. In vitro they have been described as being highly autoreactive by their propensity at being activated by syngeneic target cells expressing CD1d molecules (Bendelac et al. 2007, Annual Rev. Immuno., 25:297; Park et al., 1998, J. Immunol., 160:3128). This phenomenon can be illustrated by the activation of Vα14 NKT hybridoma cell DN32.D3 against RBL-CD1, a CD1d positive cell line, that have been stimulated by TLF ligands. Stimulation of DN32.D3 cells and TBA.7 cells, a non-Vα14 NKT cell hybridoma, was tested against RBL-CD1 or RBL-CD1 SAP^{-/-} in which saponin expression was knocked down by interfering RNAs. The hybridoma cells were cultured in RPMI supplemented with 10% FCS, 2 mM L−glutamine, 20 mM HEPES, and non-essential amino acids. Antigen presentation assays were carried out using 5-20×10³ DC 3.2 cells or 1×10⁵ splenocytes and 4×10⁴ T cells per well in 96-well tissue culture plates in triplicates. Cell culture supernatants were collected 24 h later for determination of IL-2 concentrations using an IL-2-dependent NK cell line reporter system.

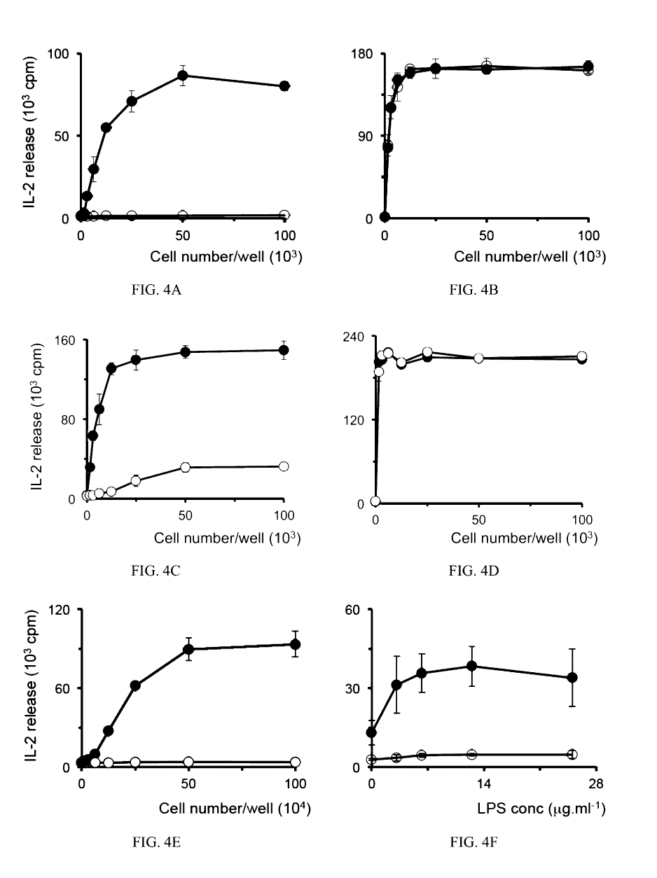

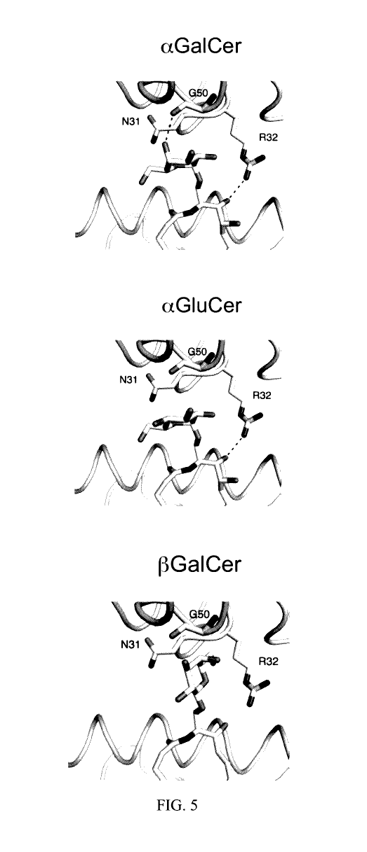

The results of the T-cell activation assay are shown in FIGS. 4C and 4D. FIG. 4C shows stimulation of the DN32.D3 cells with RBL-CD1 (filled circles) or RBL-CD1 SAP^{-/-} (open circles). FIG. 4D shows stimulation of the TBA.7 cells with RBL-CD1 (filled circles) or RBL-CD1 SAP^{-/-} (open circles). The presentation of these endogenous ligands required a competent lysosome and lipid transfer protein as shown in FIGS. 4C and 4D by the large decrease in stimulatory activity produced by the knockout of saposin in RBL-CD1 cells. The activation of DN32.D3 was blocked by anti-CD1 antibodies, such as 20H2 (data not shown).

Antibodies L317 and L363 which are specific for the complex produced by the interaction of CD1d with α-galactosylceramide (αGalCer), a ligand that is thought to be produced exclusively in non-mammalian species, were used to probe the structure of the stimulatory CD1-lipid complexes. FIG. 5 shows the predicted binding of L363 to glycosylceramides. In the crystal structure, L363 contacts αGalCer with two H-bonds—G50 interacts with the axial 4′OH while R32 is specific for the sphingosine chain (PDB ID 3UBX; FIG. 5, left panel). Modeling the interaction with αGluCer illustrated the loss of the H-bond with G50, due to equatorial rather than axial position of 4-OH, resulting in weaker L363 binding affinity (FIG. 5, middle panel). However, N31 and R32 together form a cap over the sugar and bind through VdW interactions, predominantly through N31. The upright positioning of βGalCer (modeled using the crystal structure of mCD d-sulfatide, PDB ID 2AKR) prevent L363 binding due to steric clashes (FIG. 5, right panel). The structures in FIG. 5 demonstrate that L363 does not bind p-linked glycolipids or diglycosylceramides due to obvious steric hindrances, and that L363 exhibits better binding to galactose than glucose due to a unique hydrogen bonding of the C4 hydroxyl group of the sugar with the antibody.

The addition of L317 (IgG2a) or L363 (IgG) antibodies to the T-cell activation assays was carried out as a control. The antibodies were produced in serum-free Ultradoma media (Lonza, Walkersville, Md.) in individual bioreactors. Purification of the antibodies was carried out on HiTrap protein A or G columns (GE Healthcare, Pittsburgh, Pa.). FIG. 4A shows the IL-2 production of the Vα14 expressing DN32.D3 NKT cells after a 24 h exposure to increasing numbers of RBL-CD1 cells in the presence of L363 (open circles) or control (filled circles) antibody (10 μg/ml). The non-Vα14 NKT cell hybridoma TBA.7 tested under similar conditions are shown in FIG. 4B.

FIG. 4E shows the stimulatory activity of WT thymocytes towards DN32.D3 cells tested in the presence of control (filled circles) or L363 (open circles) antibody (20 μg/ml). FIG. 4F shows the stimulation of 2×10⁴ DC3.2 cells treated for 16 hours with increasing concentrations of LPS in the presence of control (filled circles) or L363 (open circles) antibody (10 μg/ml). For all of the T-cell activation assays shows in FIGS. 4A-4F, IL-2 production was measured using the NK reporter cell line from triplicate wells. Experiments shown in FIGS. 4A-4F are representative of at least 5 separate individual experiments.

Surprisingly, both L317 and L363 antibodies efficiently blocked the activation of DN32.D3 by RBL-CD1, thymocytes and TLR-activated DCs, whereas they did not affect the activation of non-Vα14 NKT cells, such as TBA7 (see FIGS. 4A and 4B). Because the specificity of antibodies is so exquisite, the results shown in FIGS. 4A-4F strongly suggested that the ligands for Vα14 NKT cells were α-linked monoglycosylceramides.

### Example 3

### The Stimulatory Activity of Commercial β-Glucosylceramide 24:1 is not Attributable to β-Glucosylceramide

β-glucosylceramides (βGluCer) are believed to be natural endogenous ligand of NKT cells, and synthetic preparation of C12 and C24:1 βGluCer have been shown to be strong activators of type 1 NKT cells (Brennan et al., 2011, Nature Immunology, 12:1202). However, due to limitations of analytical methods for detecting and measuring lipids, the possibility of α-anomers contaminating the synthetic preparations and potentially contributing to the stimulatory activity of the preparations could not be easily ruled out.

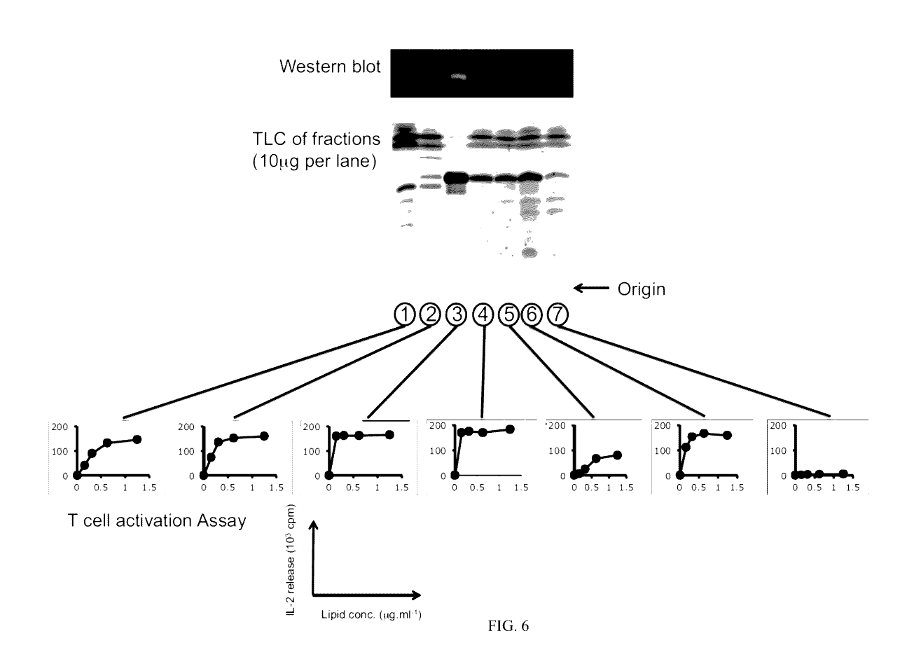

A large quantity of commercial C24:1 βGluCer and isolated 7 fractions were re-purified by normal phase chromatography performed on a Thermo Hypersil Sax column using 95:5 methanol:H₂O, 5 mM ammonium acetate loading/wash buffer and dicholormethane for elution. The seven collected fractions were analyzed by high performance TLC and immunoblotting using a rabbit anti-serum specific for β-glucosylceramide. The results are shown in FIG. 6. After lyophilization and weighing, each fraction was tested for biological activity using DC3.2 cells, a dendritic cell line expressing CD1d and susceptible to differentiation induced by TLR ligands and cytokines such as LPS and TNF, as presenting cells for DN32.D3 T cell activation (triplicates of each dilution, 2 fold dilution from 1.2 μg/ml). The same experiment was repeated twice with similar results. Maturation of the DC3.2 cells was carried out over periods of 16-24 h. Beyond 24 h, the capacity of the matured DC3.2 cells to stimulate NKT cells in a way that is sensitive to blocking with L363 or L317 antibodies was found to diminish.

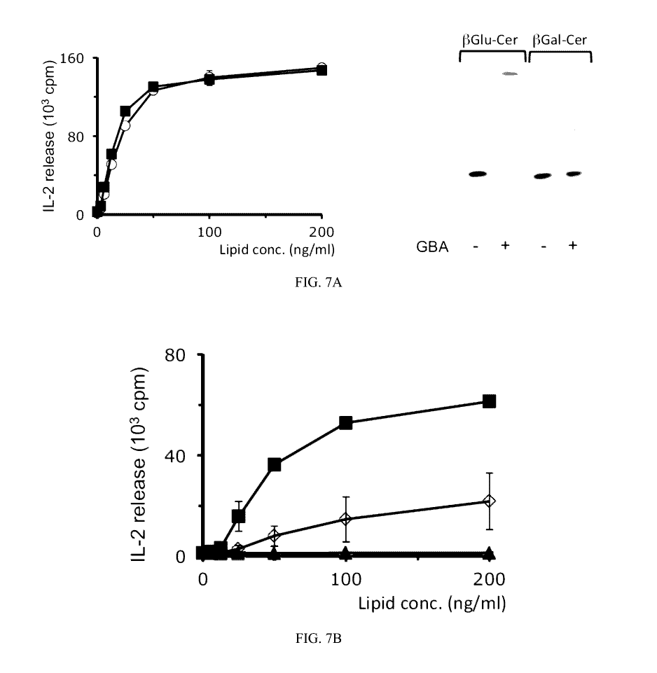

Of the collected fractions tested for the presence of βGluCer by immunoblot, only fraction 3 tested positive (FIG. 6). However, in a T-cell activation assay, 6 of the 7 fractions were found to be stimulatory. Contamination of the commercial C24:1 βGluCer preparation with α-anomers was confirmed by enzymatic digestion of fraction 3 with recombinant acid glucosylceramidase (GBA), an enzyme that is specific for βGluCer. β-glucosylceramide was digested with recombinant GBA for 2 h at 37° C. and analyzed by TLC (FIG. 7A) and functionally for its ability to stimulate DN32.D3 NKT cells when presented by WT splenocytes (10⁵ cells/well). Stimulatory activity was not changed after (squares) as compared to before (circles) digestion. FIG. 7B shows the stimulatory activity of commercial β-glucosylceramide was blocked by L363 (diamondsX 10 μg/ml) and 20H2 (triangles)(5 μg/ml) but not control (squares) anti-MHC class II antibodies MK6 (anti-I-A^{d}, IgG2a) and 14.4.4s (anti-I-E^{k}, IgG2a).

The data in FIGS. 7A and 7B demonstrates that the enzymatic removal of glucose from β-GluCer does not affect the stimulatory activity of the commercial C24:1 βGluCer. Furthermore, most of the remaining activity could be blocked by L317 or L363, suggesting that an α-anomer species was the stimulatory contaminant. In addition, L363 antibody was also unable to block the stimulation of DN32.D3 T cells by DCs loaded with isoglobotrihexosylceramide (iGb3), a known agonist of NKT cells (Zhou et al., 2004, Science, 306:1786) (data not shown).

To confirm unambiguously these data, binding of L363 antibody by surface plasmon resonance (SPR) against a large series of α and β gluco- and galactosylceramides was examined. A listing of the analyzed lipids and structure of the lipids is shown in Table 1.

| | Affinity | |
|---|---|---|
| Lipid | (K_{diss}/Kₐₛₛ = K_{d}) | |
| name | s⁻¹/M.s⁻¹ = M | Structure |
| αGalCer | 0.0083/8.57E⁺⁴ = 9.67E⁻⁸ | 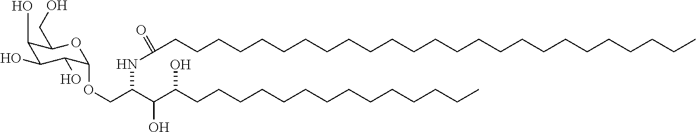 [SMILES: CCCCCCCCCCCCCCCCCCCCCCCCCC(=O)N[C@@H](COC1OC(CO)C(O)C(O)C1O)C(O)[C@H](O)CCCCCCCCCCCCCC] |
| αGalCer24:1 | 0.05/1.42E⁺⁵ = 3.50E⁻⁷ | 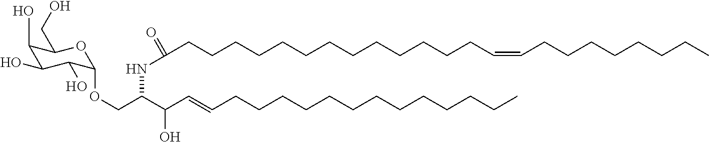 [SMILES: CCCCCCCC/C=C\CCCCCCCCCCCCCC(=O)N[C@@H](COC1OC(CO)C(O)C(O)C1O)C(O)/C=C/CCCCCCCCCCCCC] |
| βGalCer24:1 | N.M. | 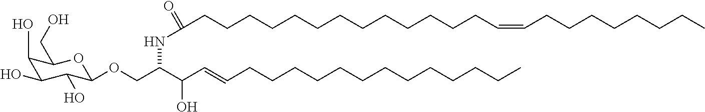 [SMILES: CCCCCCCC/C=C\CCCCCCCCCCCCCC(=O)N[C@@H](COC1OC(CO)C(O)C(O)C1O)C(O)/C=C/CCCCCCCCCCCCC] |
| αpsychosine | 0.021/9.43E⁺⁵ = 2.23E⁻⁶ | 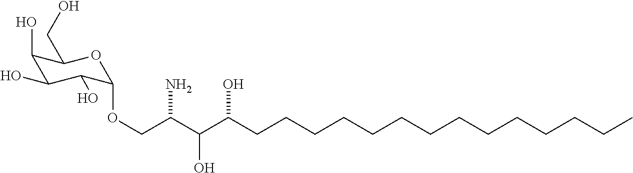 [SMILES: CCCCCCCCCCCCCC[C@@H](O)C(O)[C@@H](N)COC1OC(CO)C(O)C(O)C1O] |
| βpsychosine | N.M. | 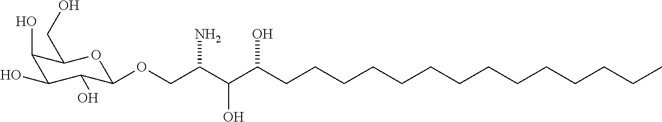 [SMILES: CCCCCCCCCCCCCC[C@@H](O)C(O)[C@@H](N)COC1OC(CO)C(O)C(O)C1O] |
| αGluCer | 0.4711/9.37E⁺⁴ = 5.02E⁻⁶ | 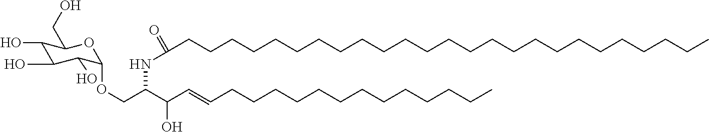 [SMILES: CCCCCCCCCCCCC/C=C/C(O)[C@H](COC1OC(CO)C(O)C(O)C1O)NC(=O)CCCCCCCCCCCCCCCCCCCCCCCCC] |
| βGluCer24:1 | N.M. | 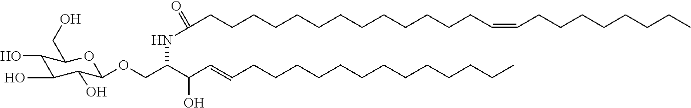 [SMILES: CCCCCCCC/C=C\CCCCCCCCCCCCCC(=O)N[C@@H](COC1OC(CO)C(O)C(O)C1O)C(O)/C=C/CCCCCCCCCCCCC] |
| αgluco- psychosine | 0.1152/4.59E⁺³ = 8.14E⁻⁶ | 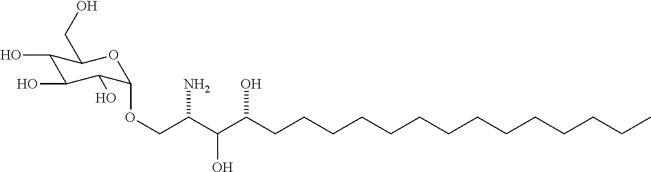 [SMILES: CCCCCCCCCCCCCC[C@@H](O)C(O)[C@@H](N)COC1OC(CO)C(O)C(O)C1O] |
| βgluco- psychosine | N.M. | 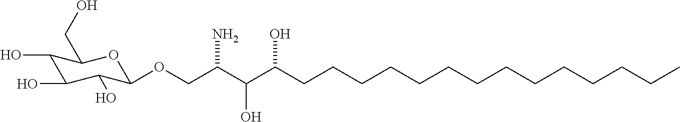 [SMILES: CCCCCCCCCCCCCC[C@@H](O)C(O)[C@@H](N)COC1OC(CO)C(O)C(O)C1O] |

A Biacore T200 instrument (available from GE Healthcare, Pittsburgh, Pa.) was used for SPR measurements. Measurements were performed using single cycle protocols to avoid repeated use of regeneration buffer on the immobilized ligands. Immobilization of target antibodies was carried out using classical amine coupling chemistry. 250 to 1,000 RU of antibody was immobilized in each flow cell. All mCD1-lipid complexes were purified after loading to ensure maximal homogeneity and avoid the presence of small amounts of aggregated material. Concentrations ranging from 1 to 10 mM were used for each CD1-lipid complex. Flow cell one was used as a negative control and used for subtraction from experimental flow cells (L363 antibody—control antibody). Global analysis of subtracted sensorgrams was carried out using the T200 analysis software.

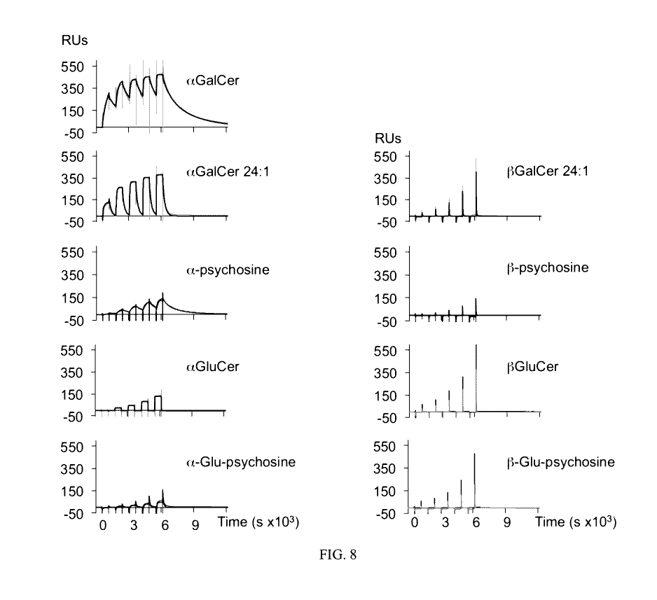

As shown in FIG. 8 and Table 1, all α-linked monoglycosyl species demonstrated measurable binding and a strong preference for galactose over glucose. Affinity constants for L363 for each of the analyzed lipids bound to CD1d is shown in Table 1. Binding of p-linked glycolipids-CD1 complexes could not be detected for either L317 or L363 antibody. Diglycosylceramides such as αGal(α1-2)Galactosylceramide and trihexosylceramide such as iGb3 loaded into CD1d, also exhibited no ability to bind the same antibodies (not shown). Interestingly, L363 and L317 were found to bind CD1 loaded with α-lyso-galactosylceramide (α-psychosine), and α-lyso-glucosylceramide (α-glucosyl-psychosine), two compounds that are potent stimulators of NKT cells in vitro and in vivo.

### Example 4

### Direct Isolation of Natural Endogenous Ligands

Because it is likely that the endogenous ligands are made in a very small amount, we attempted to isolate the ligands from NKT cell lines that could be grown in large quantities instead of using animal tissue. For enrichment and purification, endogenous ligands were immunoprecipitated with L363 or L317 antibody in the presence of a detergent, sodium 3-[(2-methyl-2-undecyl-1,3-dioxolan-4-yl)methoxy]-1-propanesulfonate, that was compatible with MS analysis and unlikely to compete with CD1-bound lipids efficiently. Sodium 3-[(2-methyl-2-undecyl-1,3-dioxolan-4-yl)methoxy]-1-propanesulfonate was shown in vitro to not bind CD1d to any measurable extent (data not shown). 2×10⁹ cells were harvested, washed 3 times in PBS and incubated with 50 μg of antibody for 2 h at room temperature on a rotating wheel, before being pelleted and lysed in 100 mM Tris pH7.5, 150 mM NaCl, 0.1% Rapigest (Waters). Cell debris were removed by centrifugation at 15,000 rpm for 30 min at 4° C. and 10 μg of L363 antibody was added. After 2 h, the antibody was recovered using protein-A Sepharose beads (GE Healthcare, Pittsburgh, Pa.). After extensive washes in phosphate buffer saline, lipids were directly extracted with a chloroform/methanol 2:1 mix by 2 min vortexing followed by centrifugation. Extraction was performed twice.

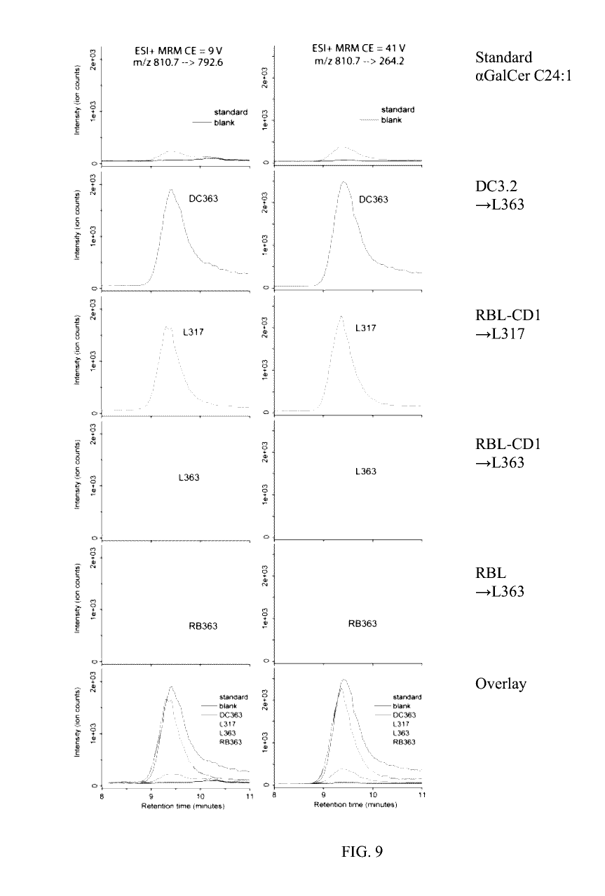

The lipid content of the L363 and L317 antibody immunoprecipitations from DC3.2 and RBL-CD1 cells (2×10⁹ cells) were analyzed by MRM mass spectrometry. Ionization transition profiles were defined for αGalCer, αGalCer C24:1 (FIG. 9), psychosine and phyto-psychosine. Untransfected RBL cells were used as a negative control (5×10⁹ cells). For MRM, an Agilent (Santa Clara, Calif.) 1200 UPLC system was coupled to a 6490 triple quadrupole mass spectrometer using multiple reaction monitoring for enhanced sensitivity and selectivity. Samples were loaded onto an Agilent 300SB C8 2.1×100 mm column and separated using the following gradient: T=Omin 80% A to T=12 min 5% A. Stop time was at 17 min, with a 4.5 min re-equilibration time between runs. Mobile phase A consisted of 95:5 H2O:MeOH, and B of 65:30:5 IPA:MeOH:H2O. Flow rate=200 μl/min and 5 μl of each sample and standard was injected. The 6490 triple quad mass spectrometer was equipped with a jet stream source with the following settings: gas temp=200° C., gas flow=14 L/min, nebulizer pressure=25 psi, sheath gas temp=350 C, sheath gas flow=10 L/min. Capillary voltage was maintained at 3500V, and nozzle voltage was set to 2000V. Two transitions were monitored to increase confidence: m/z 810.7->792.6 with collision energy set to 9V and 810.7->264.2 with collision energy at 41V. Fragmentor voltage was kept constant at 380V. Data was collected in positive ion mode.

Using RBL-CD1 cell line as starting material, the presence of a C24:1 glycosylceramide (mass of 801 Daltons) was demonstrated in six independent experiments whereas only traces of lysoglycosylceramide (mass of 462 Daltons) could be found in two experiments (FIG. 9). Similar compounds were isolated from the DC line DC3.2. Both L363 and L317 antibodies isolated the same molecular species. The stimulatory nature of the isolated glycolipids could not be tested directly because of the very limited quantities isolated from 2×10⁹ cells. The anomeric nature of the isolated compound as a α-linked ceramide could not be probed directly by mass spectrometry (isobaric species).

The direct demonstration of a C24:1 monoglycosylceramide species by MRM MS did not eliminate the possibility that other stimulatory species were also associated to CD1d. Indeed, all of the tested α-glycosylceramides with or without acyl chains (psychosines) were found to have stimulatory activity but much lower affinity for CD1d molecules and the L363 antibody (FIG. 8). This data suggested that glucosylceramides and lysoceramides were most likely lost during the immunopurification procedure.

### Example 5

### Synthesis and Degradation of the Natural Ligands

For an innate lymphocyte, NKT cells are intended to be activated rapidly and briefly to avoid stunning and anergy (Wilson et al., 2003, Proceedings of the National Academy of Sciences of the United States of America, 100:10913). Therefore, the time scale of the de novo synthesis of glycosylceramides is likely not the most appropriate mechanism to address such a requirement (Hayes and Jungalwala, 1976, Biochemical Journal, 160:195). An alternative mechanism would be to mobilize a pre-existing pool or to limit the degradation of a compound produced in very limited quantities and degraded efficiently. The synthesis of α-linked glycosylceramides by mammalian cells has largely been ruled out based on theoretical arguments favoring a S_{N}2-like over a S_{N}1 reaction in the ligation of UDP-α glucose or galactose to ceramide by glycosyltransferases (Lairson et al., 2008, Annual Rev. Biochem., 77:521). This prediction has been largely confirmed experimentally. However, the possibility that small amounts of α-linked glycosylceramides could be produced by these enzymes, or that under physiologically unique conditions such as the acidic environment of the lysosome, α-linked species could be produced spontaneously or with the help of anomerases, has not previously been considered.

In order to demonstrate the production of α-linked species in cells, DC3.2 cells were treated with competitive inhibitors of α-glucosidases and α-galactosidases. The specificity of these inhibitors has been previously studied in vitro on recombinant proteins and cell lines. In the experiments, two inhibitors that have been previously tested in human, 1-deoxygalactonojirimycin and 1-deoxygluconojirimycin (Wennekes et al., 2009, Angew Chem. Int. Ed. Engl., 48:8848; Ishii et al., 2009, J. Pharmacol. Exp. Ther., 328:723; Khanna et al., 2012, PLoS One, 7:e40776 (2012)) were used at concentrations (0.5 μM and 2.0 μM respectively) found to be optimal for specifically inhibiting α-glycosidases. DC3.2 cells were treated for 16 h with 2 ng/ml recombinant TNFα and used to stimulate DN32.D3 NKT cells in the presence of the enzymatic inhibitors.

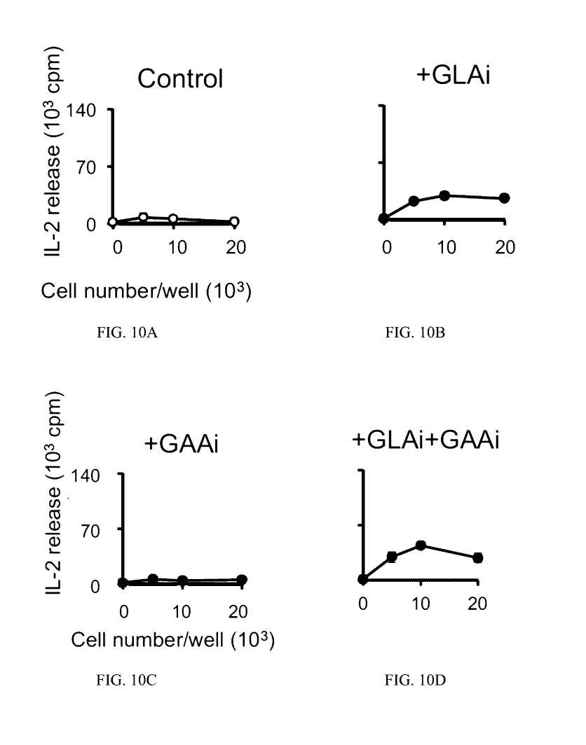

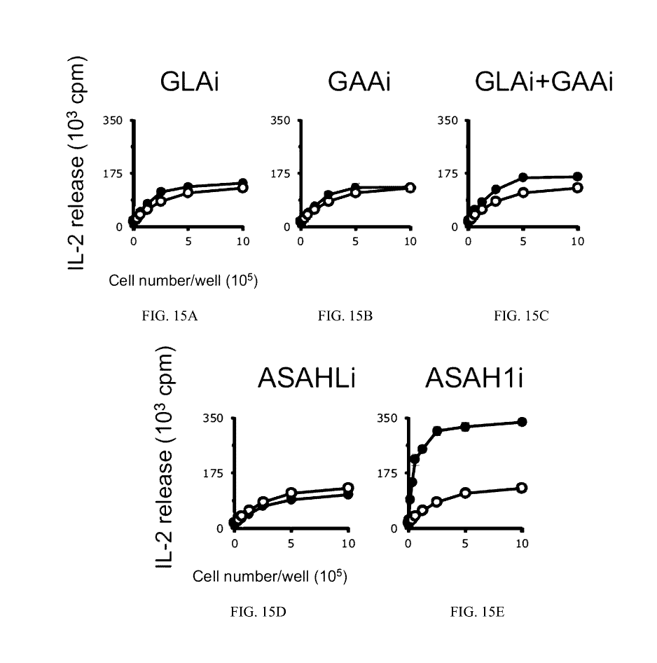

FIGS. 10A-D show the results for non-differentiated DCs and FIGS. 14A-D show the results for LPS-treated DCs. Inhibition of α-galactosidase activity with 1-deoxygalactonojirimycin induced or increased a robust stimulation of NKT cells by non-differentiated and LPS-treated DCs, respectively. In both instances, the addition of 1-deoxygluconojirimycin to block α-glucosidase did not result in significant increase in stimulatory activity.

However, in the context of cytokine (TNFα) stimulation of the undifferentiated DCs (FIGS. 11A-C), both inhibitors had similar effects and did increase stimulation of NKT cells. These results were reproduced in a large set of independent experiments (at least five for each condition) and suggested that in dendritic cells, the availability of α-glycosylceramides was directly controlled by catabolic enzymes and that their regulation was highly context and differentiation dependent.

α-glucosidase (GAA) deficiency is responsible for the lysosomal storage disease called Pompe disease and has not been studied yet in the context of NKT biology (Bijvoet et al., 1998, Hum. Mol. Genet., 7:53). In contrast, α galactosidase (GLA) deficiency, the cause of Fabry disease, has been characterized in the mouse and human with respect to NKT functions (Darmoise et al., 2010, Immunity, 33:216; Pereira et al., 2013, Mol. Genet. Metab., 108:241). The phenotype and function of the Fabry NKT cells has been found to correlate with a profile of hyper-stimulation and hyper-responsiveness and to the presence of an increased amount of self-ligands for NKT cells at the surface of selecting thymocytes and peripheral antigen presenting cells (Darmoise et al., 2010, Immunity, 33:216; Pereira et al., 2013, Mol. Genet. Metab., 108:241). The interpretation of these data in the context of GLA being exclusively specific for terminal α-galactose has not allowed the identification of the potential endogenous ligands. We therefore expressed recombinant functional GLA and tested it in vitro on a series of α-galactosylceramides.

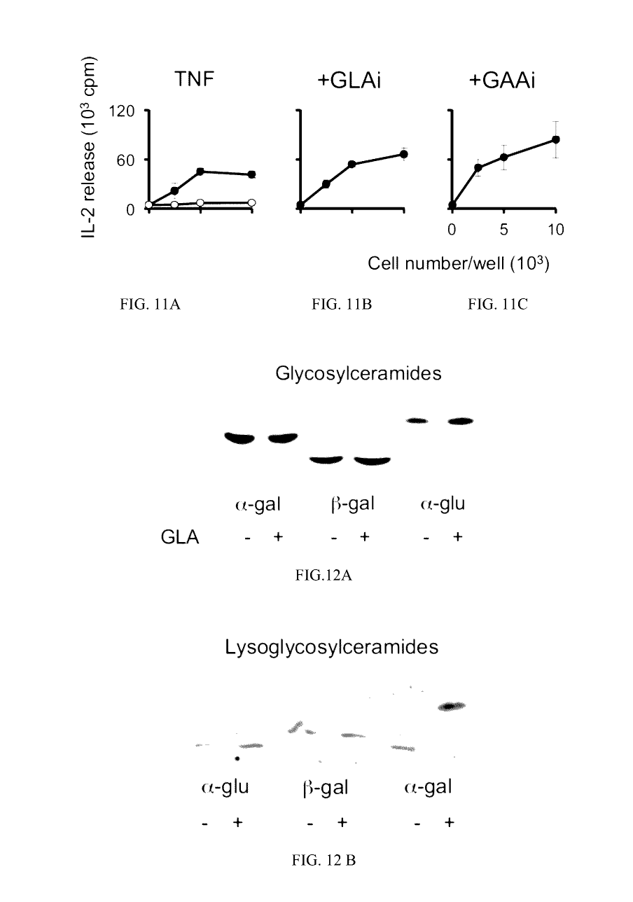

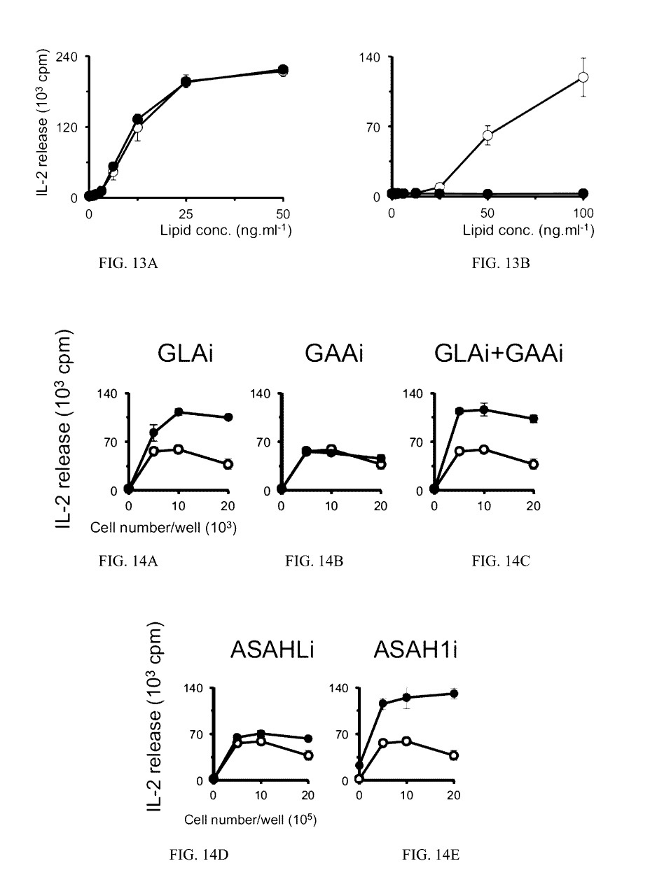

Full length murine cDNA (1-1260) was modified to add a C-terminal histidine tag by PCR and cloned into a fly expression vector. The GLA was expressed in serum-free media and purified by a succession of Ni-NTA and ion exchange chromatography. Enzymatic activity of the purified recombinant protein was evaluated on synthetic commercial glucosylceramide (Avanti Polar Lipids, Alabaster, Ala.) and synthetic commercial globotriaosylceramide (Matrey, Pleasant Gap, Pa.) followed by TLC analysis. High performance TLC was used to separate glycosyl (FIG. 12A) and lysoglycosylceramides (FIG. 12B) before and after digestion with recombinant GLA. The lipids were visualized using a Cerium Ammonium Molybdate stain. Samples from α-galactosyl (FIG. 13A) and α-psychosine (FIG. 13B) were tested for their stimulatory ability towards DN32.D3 NKT cells before (empty circles) and after (filled circles) digestion with recombinant GLA. DC3.2 cells (20,000 cells/well) were used as antigen presenting cells. As shown in FIGS. 12 and 13, the only cleavable species was α-psychosine, a substrate that has not been previously examined in the context of GLA.

The effect of 1-deoxygalactonojirimycin on DCs, the phenotype of the Fabry mouse and the in vitro activity of recombinant GLA can now be explained by a unifying reasonable interpretation: GLA controls the amount of available α-galactosylceramides, one of the endogenous NKT ligands. A similar phenotype would be expected in the GAA deficient mouse. Since GLA can only cleave galactose on α-psychosine, its activity will depend on the production of lysoceramides. In vivo, this catabolic step is controlled by lysosomal ceramidases. To our knowledge, the lysosome retains two known ceramidases that produce lyso-gluco and lysogalactosylceramides: acid ceramidase (ASAH1) (Park and Schuchman, 2006, Biochimica et biophysica acta, 1758:2133), whose deficiency is embryonically lethal in mice (Eliyahu et al. 2007, FASEB J., 21:1403), and N-acylamidehydrolase (NAAA or ASHL), a poorly characterized enzyme that is homologous to ASAH1 (Tsuboi et al., 2005, J. Biol. Chem., 280:11082).

In the absence of a suitable animal model, enzyme inhibitors were used to assess the role of ceramidases in the control of NKT ligands. 1-Deoxynojirimycin, N-[(1R,2R)-2-hydroxy-1-(hydroxymethyl)-2-(4-nitrophenyl)ethyl]-tetradecanamide (NMAPPD), a well-characterized specific inhibitor of ASAH1 exhibited too much toxicity over a 24 h assay to be usable (toxicity was evaluated by measuring viability and αGalCer presentation to DN32.D3 cells after treatment). However, carmofur (1-hexylcarbamoyl-5-fluorouracil; Sigma-Aldrich, St. Louis, Mo.) a drug with high specificity for ASAH1 (Realini et al., 2013, Sci. Rep., 3:1035) was usable in the same assay with very limited cell toxicity. In the T-cell activation assays, DC3.2 cells were differentiated with LPS and treated with inhibitors of α-glycosidases (GLAi and/or GAAi), 1-deoxygalactonojirimycin (0.5 μM) and 1-deoxygluconojirimycin (2.0 μM), respectively, or ceramidase inhibitors (NAAA/ASAHLi(27) (20 μM) or AC/ASAH1i, carmofur (1.0 μM) for 24 h and used to stimulate DN32.D3 NKT cells. The results are shown in FIGS. 14A-E. A control (open circles) was included for comparison with each inhibitor (filled circles).

As shown in FIGS. 14A-E, the inhibition of ASAH1 induces a significant increase in stimulatory activity towards NKT cells, whereas the inhibition of NAAA with a specific inhibitor resulted in a very limited but reproducible increase in stimulatory activity of untreated or LPS-treated DCs. These inhibition experiments favor a model in which degradation occurs in two successive steps: (1) removal of the acyl chain of α-ceramide followed by (2) the removal of the sugar by an α-glycosidase. Although it appears ASAH1 has a preponderant role in assuming the first step, it should be noted that the inhibitor of ASAHL used in the experiments had a very high specific activity (10-200M) and the limited effects that were observed could be associated to the low specific activity. In any case, it is clear from the limitation of these studies that the control of the availability of NKT cell endogenous ligands in the ceramidase pathway requires highly specific and active inhibitors.

Altogether these experiments revealed the existence of a new important catabolic pathway that controls the availability of natural NKT ligands and utilizes a two-step enzymatic process to degrade α-glycosylceramides efficiently.

### Example 6

### Ex Vivo Effect of CD1-α-Galactosylceramide Blockade

Using the same enzyme inhibitors as in Example 5, the stimulatory activity of thymocytes towards DN32.D3 T cells was examined in the presence or absence of the inhibitors of glycosidases and ceramidases (FIGS. 15A-E). A control (open circles) was included for comparison with each inhibitor (filled circles). As in the case of DCs, glycosidase inhibitors modulated the amount of cell surface NKT ligands in a reproducible but much more limited way. In this instance, it appeared that both GLA and GAA controlled the amount of ligand presented by thymocytes. The inhibition of ASAH1 by carmofur had the most dramatic impact on the stimulatory activity of thymocytes with a 2.5 fold increase (FIG. 15E), whereas ASAHL inhibition had no effect (FIG. 15D).

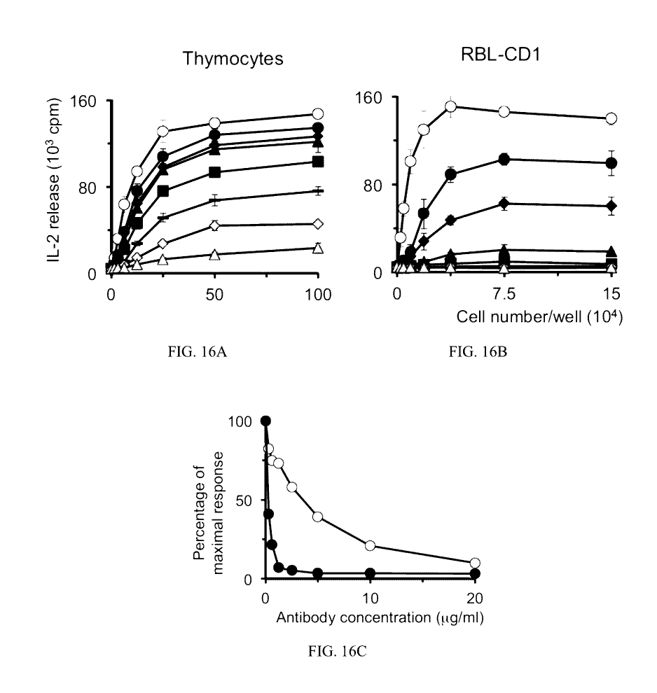

The likely presence of both α-glucosyl and α-galactosylceramides in thymocytes suggested by the inhibition experiment was supported by titration of the inhibitory activity of L363 antibody (FIGS. 16A-C). In the titration experiment, thymocytes (FIG. 16A) or RBL-CD1 cells (FIG. 16B) were used as antigen presenting cells. The RBL-CD1 cells and thymocytes were calibrated for stimulation towards DN32.D3 cells and compared side by side against increasing concentration of L363 antibody. Seven two-fold dilutions were tested from 20 μg/ml down. Percentage inhibition was plotted as percentage of maximal response (100%) for RBL-CD1 (black symbols) and thymocytes (open circles).

With increasing concentration of antibody, stimulatory activity of both cell types diminished but the IC₅₀ was ˜5 μg/ml for thymocytes and less than ˜0.325 μg/ml for RBL-CD1 cells (FIG. 16C). This difference could not be explained by antibody target density since RBL-CD1 was more potent than thymocytes, but only by differential affinity of the antibody on the two cell types tested. Given the results of the SPR measurements in Example 3, it was determined that on thymocytes, CD1d-α-glucosylceramides were presented and required higher concentration of antibody to be blocked, whereas RBL-CD1 expressed mainly the higher affinity CD1-α-galactosylceramide complexes. The titration experiment did not exclude that α-galactosylceramide was also being presented by thymocytes. However, if α-galactosylceramide was being presented by thymocytes it was in limited quantities as compared to α-glucosylceramide.

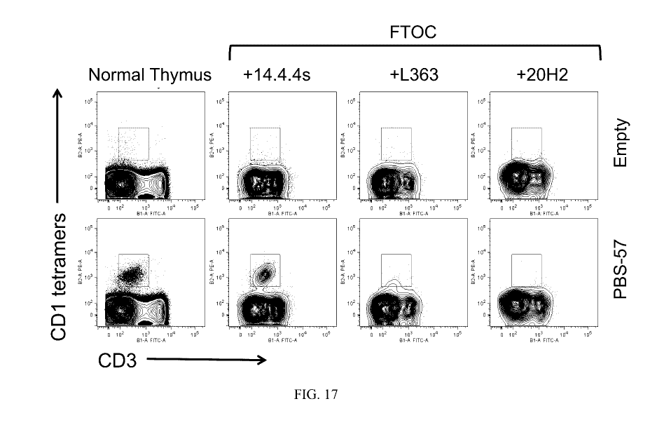

To confirm the in vitro observations, the L363 antibody was tested on fetal thymic organ cultures (FTOC) and compared to a negative control antibody (14.4.4s) and a positive control anti-CD1 antibody (20H2). The results are shown in FIG. 17. Embryonic day 14.5 fetal thymic lobes were harvested from timed pregnant C57BL/6J mice and cultured on nitrocellulose filters (Whatman) placed on a sponge (Gelfoam size 4; Upjohn Pharmacia, Peapack, N.J.). Lobes were cultured for 18 days in 0.5 mL DMEM (containing 10% FCS, 2 mM L-glutamine, 20 mM HEPES, non-essential amino acids and antibiotics) per well on 48-well tissue culture plates. Antibodies were added to the media throughout the culture period at a concentration of 60 μg/mL. Media was changed every 3 days. Cells were harvested by mechanical disruption of the thymic lobes, passaged through a 70 μm cell strainer and stained for flow cytometry (Pellicci et al., 202, J. Exp. Med., 195:835).

NKT cells were quantified using CD1d tetramers, empty or loaded with PBS-57. The CD1d tetramers were produced as described, for example, in U.S. Pat. No. 8,227,581. Tetramer and antibody staining was performed on single cell suspensions prepared from adult thymi and FTOC lobes. Organs were collected in cold flow buffer (FB; PBS containing 2% FCS/2 mM EDTA) and were passed through a 70 μm cell strainer to obtain a single cell solution. Samples were depleted of erythrocytes using 0.165M NH4Cl in water. Samples were washed twice and treated with Fc Block (BD Biosciences, San Jose, Calif.) and 0.5 mg/ml avidin (Sigma-Aldrich, St. Louis, Mo.) in FB at room temperature for 10 min. Cells were then washed with FB and stained with CD1d/Empty or CD1d/PBS-57 tetramers at room temperature for 30 min. Anti-CD3ε and anti-B220 (BD Biosciences-Pharmingen, San Diego, Calif.) were directly added, and staining continued for another 20 min. Samples were washed twice in FB and propidium iodide was added for dead cell exclusion. Samples were acquired on a MACSQuant analyzer using MACSQuantify software (Miltenyi Biotec, San Diego, Calif.) and analyzed with FlowJo software (Tree Star Inc., Ashland, Oreg.).

At day 18 of culture, d14.5 thymi treated with L363 antibody did not contain detectable NKT cells as measured using CD1d tetramers loaded with PBS-57 (FIG. 17). Respective percentages of CD1-PBS57 positive cells were 0.27, 8.02, 1.14, and 0.27% for adult thymus, 14.4.4s, L363, and 20H2, respectively. Similar results were obtained on four other thymic lobes. In addition to confirming the in vitro observations, the FTOC data demonstrated the usefulness of manipulating NKT cell production and numbers with antibodies to treat autoimmune and chronic infectious conditions and/or evaluate the effects of NKT cells in autoimmune and chronic infectious conditions.

### DISCUSSION

The biochemistry of lipids has been limited by the lack of sensitivity of analytical techniques. Examples 2-6 addressed these limitations by combining the exquisite sensitivity of T cells in biological assays with the specificity of immunoglobulins, the stereo-specificity of catabolic enzymes and techniques such as MRM mass spectrometry to identify and characterize the natural endogenous ligands of NKT cells. This approach identified α-glycosylceramides as the main endogenous ligands of NKT cells in the thymus and the periphery. From a reductionist approach, dendritic cells were found to be the most relevant cell type for NKT functions in the periphery.

The results presented in Examples 2-6 and the accompanying figures address a number of unexplained observations in the field of NKT biology. First, the presence of both α-glucosyl and α-galactosylceramides explains why removal of a single pathway, such as performed in acid galactosylceramidase knockout animals, does not eliminate NKT cells. Second, the results presented herein support catabolism as controlling the amount of NKT agonists in the phenotype of the Fabry mouse. In the Fabry mouse, even though the lysosome is dysfunctional, NKT cells are hyper-responsive in the periphery and exhibit stigmas of permanent activation. The thymic production of NKT in these mice is relatively unaffected, suggesting that glucosylceramides are equally or more important than galactosylceramides in the thymus. Third, presentation of ligands to NKT cells is controlled by degradation, allowing a very fast response in the context of inflammation and infection. A two-step degradation process was required for the complete removal of stimulatory activity since the α-glycosidase, as shown for GLA, only cleaves the sugar from lysoceramides. Fourth, the dual nature of the endogenous ligands of NKT cells is likely the basis for the tissue specificity of NKT cell subpopulations. The balance between glucosyl and galactosyl species as seen between thymocytes and DCs, is a general mechanism that favors the local expansion of NKT cells tuned for the recognition of one or the other ligand. Finally, the basal production of α-glycosylceramide appears to be controlled, at least in part, by a mechanism in which a small amount of α-linked ceramide is produced enzymatically and efficiently degraded to avoid NKT cell activation. In conclusion, Examples 2-6 revealed the existence of a metabolic and degradative pathway in glycolipid metabolism that produces α-linked glycolipids and provides new approaches in the utilization of NKT cells and NKT cell agonists in immunotherapy.

While the compositions and methods of this disclosure have been described in terms of exemplary embodiments, it will be apparent to those skilled in the art that variations may be applied to the compositions and methods and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure. In addition, all patents and publications listed or described herein are incorporated in their entirety by reference.

All publications, patents and patent applications referenced in this specification are indicative of the level of ordinary skill in the art to which this invention pertains. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

It also is specifically understood that any numerical value recited herein includes all values from the lower value to the upper value, i.e., all possible combinations of numerical values between the lowest value and the highest value enumerated are to be considered to be expressly stated in this application.

1. A compound represented by formula I: 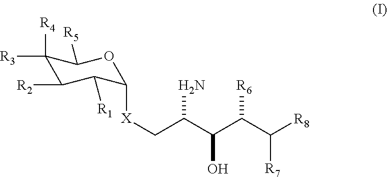
wherein:
X is O, S, or CH₂,
R₁, is —OR₉, wherein R₉ is —H, —SO₃H, or a pharmaceutically acceptable salt;
R₂ is —OH, —SO₃H, —OSO₃H, —PO₄, —PO₄H, —COOH, or a pharmaceutically acceptable salt;
R₃ is —H if R₄ is —OR₉ or R₃ is —OR₉ if R₄ is —H;
R₅ is —C(O)R₆ wherein R₆ is —OH, —OSO₃H, or a pharmaceutically acceptable salt thereof or —CH₂OR₉;
R₆ is —H, —OR₉, or forms a double bond with R₇;
R₇ is —H or forms a double bond with R₆; and
R₈ is a saturated or unsaturated hydrocarbon having from about 5 to about 15 carbons.

2. The compound of claim 1, wherein the compound has the following structure: 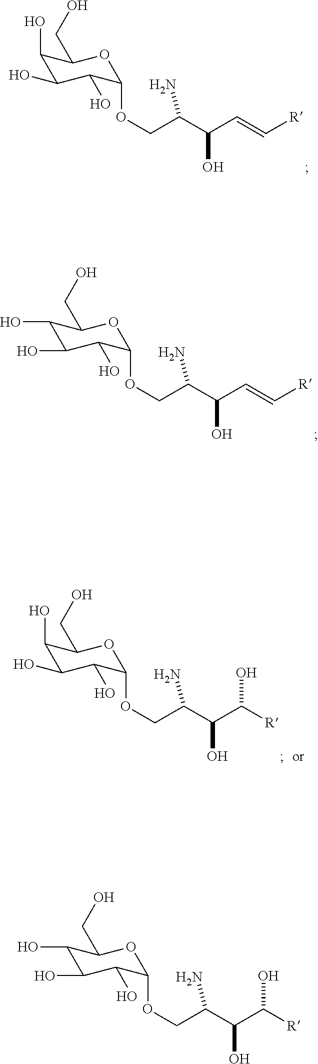
and R′ is a saturated or unsaturated hydrocarbon having from about 5 to about 15 carbons.

3. The compound of claim 1, wherein the compound has the following structure: 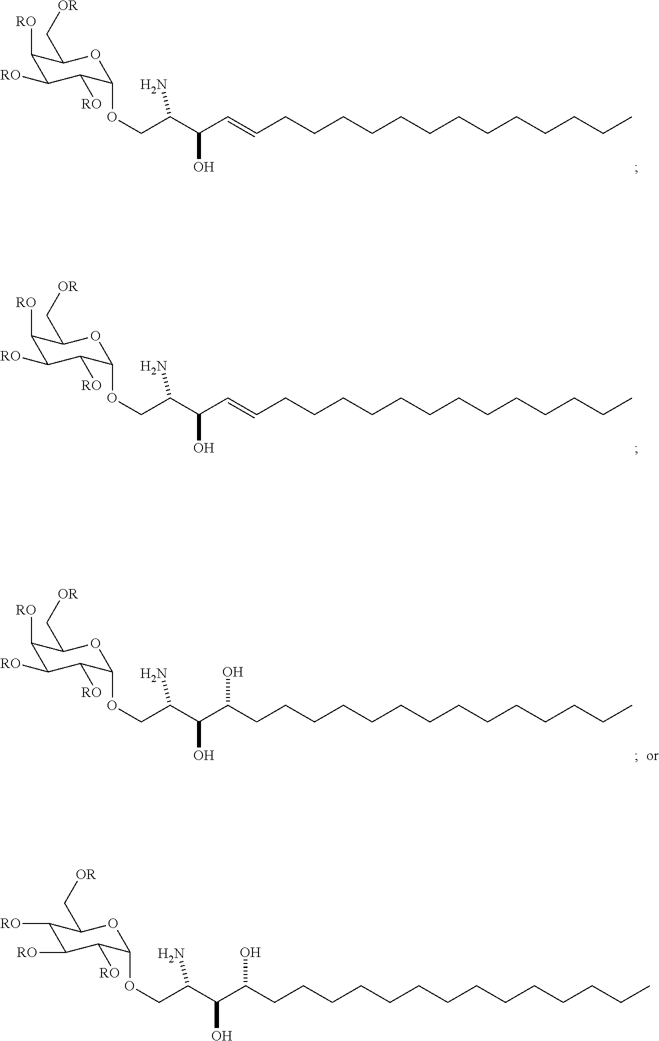
and R is independently —H, —OSO₃, or a pharmaceutically acceptable salt.

4. The compound of claim 1, wherein the compound is:

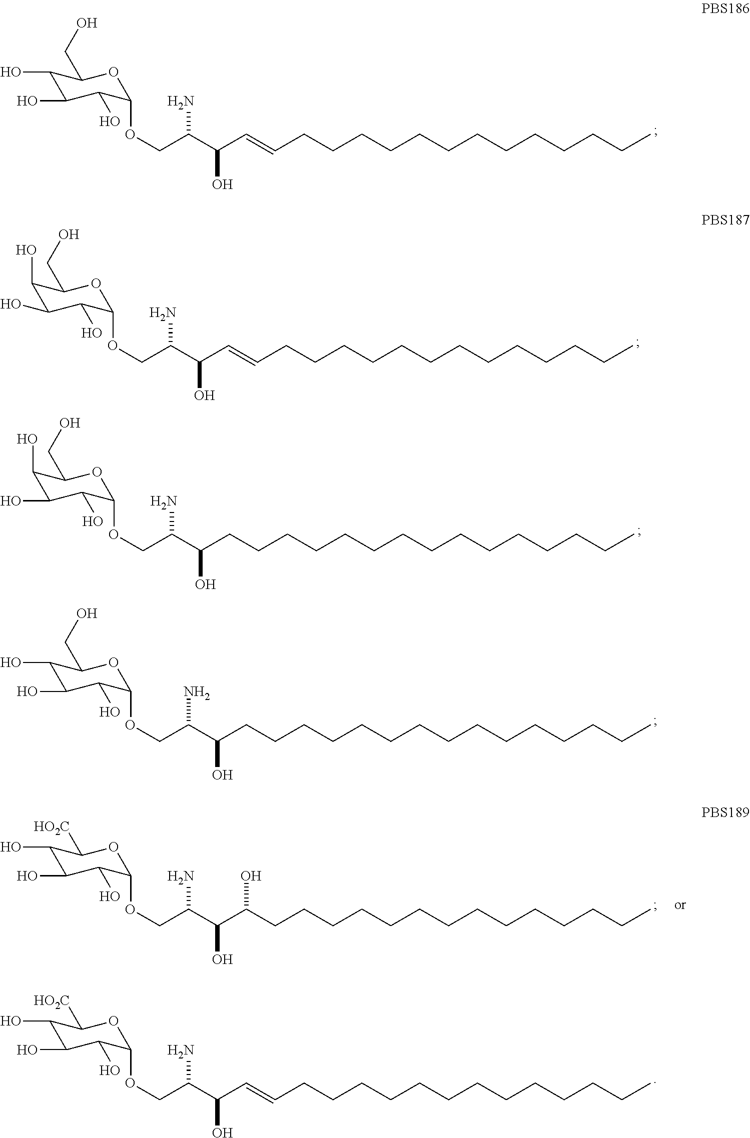 [SMILES: CCCCCCCCCCCCC/C=C/[C@@H](O)[C@@H](N)COC1OC(CO)C(O)C(O)C1O ; CCCCCCCCCCCCC/C=C/[C@@H](O)[C@@H](N)COC1OC(CO)C(O)C(O)C1O ; CCCCCCCCCCCCCCC[C@@H](O)[C@@H](N)COC1OC(CO)C(O)C(O)C1O ; CCCCCCCCCCCCCCC[C@@H](O)[C@@H](N)COC1OC(CO)C(O)C(O)C1O ; CCCCCCCCCCCCCC[C@@H](O)[C@@H](O)[C@@H](N)COC1OC(C(=O)O)C(O)C(O)C1O ; CCCCCCCCCCCCC/C=C/[C@@H](O)[C@@H](N)COC1OC(C(=O)O)C(O)C(O)C1O]

5. A compound represented by formula II: 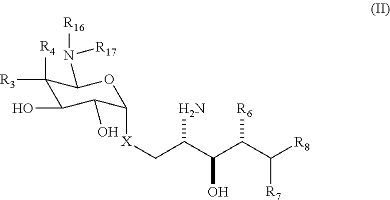
wherein:
X is O, S, or CH₂;
R₁₆ is selected from:
(i) C(O)R₁₃;
(ii) C(R₁₃)R₁₄, wherein R₁₄ is —H and R₂ forms a double bond between nitrogen and the carbon to which R₁₄ is attached;
(iii) C(R₁₃)R₁₄(R₁₅), wherein R₁₄ is H or R₁₃ and R₁₅ is —H or R₁₃; or
(iv) SO₂R₁₃;
wherein R₁₃ is halo; hydroxy, OR₉; OR₁₀; amino, NHR₉; N(R₉)₂; NHR₁₀; N(R₁₀)₂; aralkylamino; or C₁-C₂ alkyl optionally substituted with halo, hydroxyl, oxo, nitro, OR₉, OR₁₀, acyloxy, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₉, NHSO₂R₁₀, sulfate, phosphate, cyano, carboxyl, C(O)R₉, C(O)R₁₀, C(O)OR₉, C(O)NH₂, C(O)NHR₉, C(O)N(R₉)₂, C₃-C₁₀ cycloalkyl containing 0-3 R₁₁, C₃-C₁₀ heterocycyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl containing 0-3 R₁₂, or heteroaryl containing 0-3 R₁₂; or C₃-C₁₀ cycloalky, C₃-C₁₀ heterocyclyl, C₅-C₁₀ cycloalkenyl, or C₅-C₁₀ heterocycloalkenyl optionally substituted with one or more halo hydroxyl, oxo, OR₉, OR₁₀, acyloxy, nitro, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₉, NHSO₂R₁₀, sulfate, phosphate, cyano, carboxyl, C(O)R₉, C(O)R₁₀, C(O)OR₉, C(O)NH₂, C(O)NHR₁₀, C(O)N(R₁₀)₂, alkyl, haloalkyl, C₃-C₁₀ cycloalkyl containing 0-3 R₁₁, C₃-C₁₀ heterocyclyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl heteroaryl containing 0-3 R₁₂, or C₆-C₂₀ heteroaryl containing 0-3 R₁₂; or C₂-C₆ alkenyl, C₂-C₆ alkynyl, aryl, or heteroaryl optionally substituted with one or more halo, hydroxyl, OR₉, OR₁₀, acyloxy, nitro, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₁₀, sulfate, phosphate, cyano, carboxyl, C(O)R₉, C(O)R₁₀, C(O)OR₉, C(O)NH₂, C(O)NHR₉, C(O)N(R₉)₂, alkyl, haloalkyl, C₃-C₁₀ cycloalkyl containing 0-3 R₁₁, C₃-C₁₀ heterocycyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl containing 0-3 R₁₂, or C₆-C₂₀ heteroaryl containing 0-3 R₁₂;
R₁₇ is —H or C₁-C₆ alkyl;
R₃ is —H if R4 is —OH, or R₃ is —OH if R₄ is —H;
R₆ is —OH or forms a double bond with R₇;
R₇ is —H or forms a double bond with R₆;
R₈ is a saturated or unsaturated hydrocarbon having from about 5 to about 15 carbons;
each R₉ is independently a C₁-C₂₀ alkyl optionally substituted with halo, hydroxyl, alkoxy, amino, alkylamino, dialkylamino, sulfate, or phosphate;
each R₁₀ is independently an aryl optionally substituted with halo, haloalkyl, hydroxyl, alkoxy, nitro, amino, alkylamino, dialkylamino, sulfate, or phosphate;
each R₁₁ is independently halo, haloalkyl, hydroxyl, alkoxy, oxo, amino, alkylamino, dialkylamino, sulfate, or phosphate; and
each R₁₂ is independently halo, haloalkyl, hydroxyl, alkoxy, nitro, amino, alkylamino, dialkylamino, sulfate, or phosphate.

6. The compound of claim 5, wherein R₁₆ is C(O) R₁₃ where R₁₃ is C1-C12 alkyl, R₁₇ is H, R₆ is —OH or forms a double bond with R₇, and R₈ is a saturated or unsaturated hydrocarbon having from about 5 to about 15 carbons.

7. The compound of claim 5, wherein the compound is

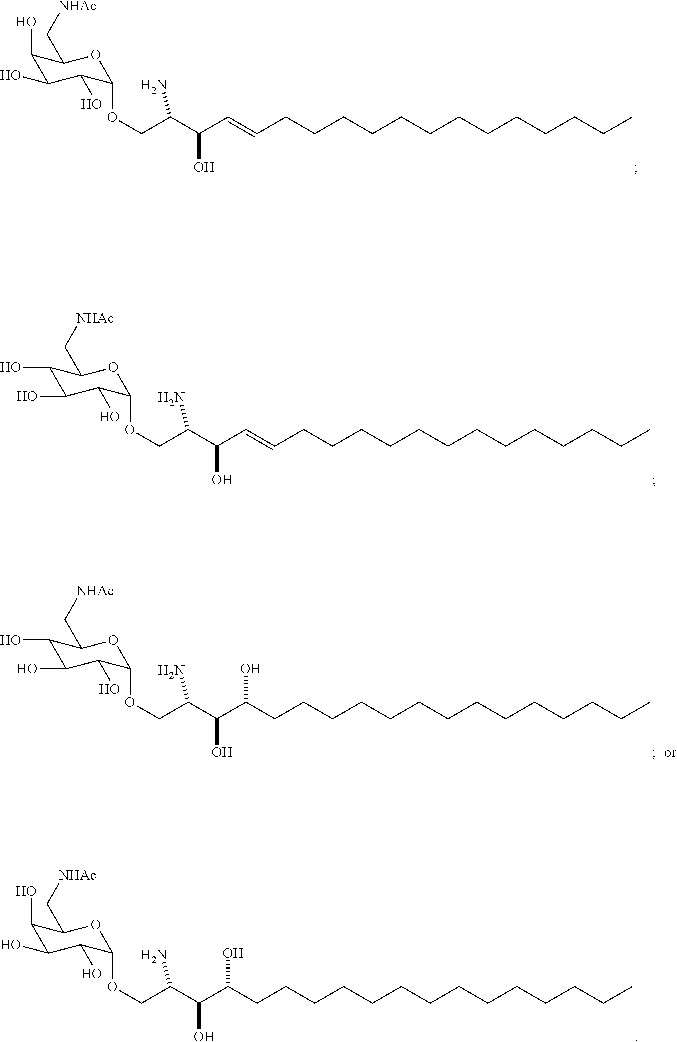 [SMILES: CCCCCCCCCCCCC/C=C/[C@@H](O)[C@@H](N)COC1OC(CNC(C)=O)C(O)C(O)C1O ; CCCCCCCCCCCCC/C=C/[C@@H](O)[C@@H](N)COC1OC(CNC(C)=O)C(O)C(O)C1O ; CCCCCCCCCCCCCC[C@@H](O)[C@@H](O)[C@@H](N)COC1OC(CNC(C)=O)C(O)C(O)C1O ; CCCCCCCCCCCCCC[C@@H](O)[C@@H](O)[C@@H](N)COC1OC(CNC(C)=O)C(O)C(O)C1O]

8. A compound represented by formula III: 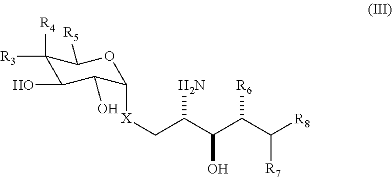
wherein:
X is O, S, or CH₂;
R₃ is —H if R₄ is —OH, or R₃ is —OH if R₄ is —H;
R₅ is —SR₁₅ or —OR₁₅;
wherein R₁₅ is C₁-C₁₂ alkyl optionally substituted with halo, hydroxyl, oxo, nitro, OR₉, OR₁₀, acyloxy, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₉, NHSO₂R₁₀, sulfate, phosphate, cyano, carboxyl, C(O)R₉, C(O)R₁₀, C(O)OR₉, C(O)NH₂, C(O)NHR₉, C(O)N(R₉)₂, C₃-C₁₀ cycloalkyl containing 0-3 R₁₁, C₃-C₁₀ heterocycyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl containing 0-3 R₁₂, or heteroaryl containing 0-3 R₁₂; or C₃-C₁₀ cycloalky or C₅-C₁₀ cycloalkenyl optionally substituted with one or more halo hydroxyl, oxo, OR₉, OR₁₀, acyloxy, nitro, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₉, NHSO₂R₁₀, sulfate, phosphate, cyano, carboxyl, C(O)R₉, C(O)R₁₀, C(O)OR₉, C(O)NH₂, C(O)NHR₁₀, C(O)N(R₁₀)₂, alkyl, haloalkyl, C₃-C₁₀ cycloalkyl containing 0-3 R₁₁, C₃-C₁₀ heterocyclyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl heteroaryl containing 0-3 R₁₂, or C₆-C₂₀ heteroaryl containing 0-3 R₁₂; or C₂-C₆ alkenyl, C₂-C₆ alkynyl, or aryl, optionally substituted with one or more halo, hydroxyl, OR₉, OR₁₀, acyloxy, nitro, amino, NHR₉, N(R₉)₂, NHR₁₀, N(R₁₀)₂, aralkylamino, mercapto, thioalkoxy, S(O)R₉, S(O)R₁₀, SO₂R₉, SO₂R₁₀, NHSO₂R₁₀, sulfate, phosphate, cyano, carboxyl, C(O)R₉, C(O)R₁₀, C(O)OR₉, C(O)NH₂, C(O)NHR₉, C(O)N(R₉)₂, alkyl, haloalkyl, C₃-C₁₀ cycloalkyl containing 0-3 R₁₁, C₃-C₁₀ heterocycyl containing 0-3 R₁₁, C₂-C₆ alkenyl, C₂-C₆ alkynyl, C₅-C₁₀ cycloalkenyl, C₅-C₁₀ heterocycloalkenyl, C₆-C₂₀ aryl containing 0-3 R₁₂, or C₆-C₂₀ heteroaryl containing 0-3 R₁₂;
R₆ is —OH or forms a double bond with R₇;
R₇ is —H or forms a double bond with R₆;
R₈ is a saturated or unsaturated hydrocarbon having from about 5 to about 15 carbons;
each R₉ is independently a C₁-C₂₀ alkyl optionally substituted with halo, hydroxyl, alkoxy, amino, alkylamino, dialkylamino, sulfate, or phosphate;
each R₁₀ is independently an aryl optionally substituted with halo, haloalkyl, hydroxyl, alkoxy, nitro, amino, alkylamino, dialkylamino, sulfate, or phosphate;
each R₁₁ is independently halo, haloalkyl, hydroxyl, alkoxy, oxo, amino, alkylamino, dialkylamino, sulfate, or phosphate; and
each R₁₂ is independently halo, haloalkyl, hydroxyl, alkoxy, nitro, amino, alkylamino, dialkylamino, sulfate, or phosphate.

9. The compound of claim 1, wherein the compound is capable of activating an NKT cell.

10. A composition comprising a compound according to claim 1.

11. The composition of claim 10, wherein the compound has the following structure: 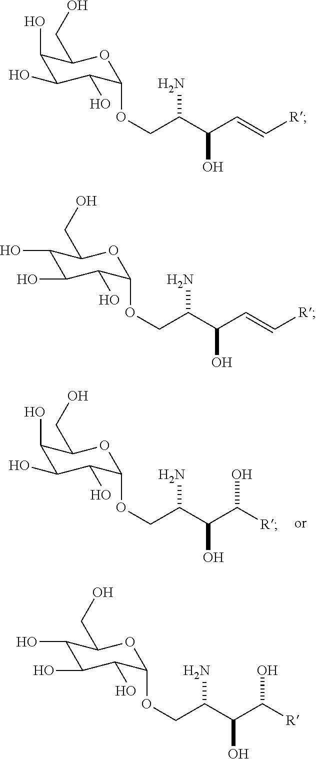
and R′ is a saturated or unsaturated hydrocarbon having from about 5 to about 15 carbons.

12. The composition of claim 10, wherein the compound has the following structure: 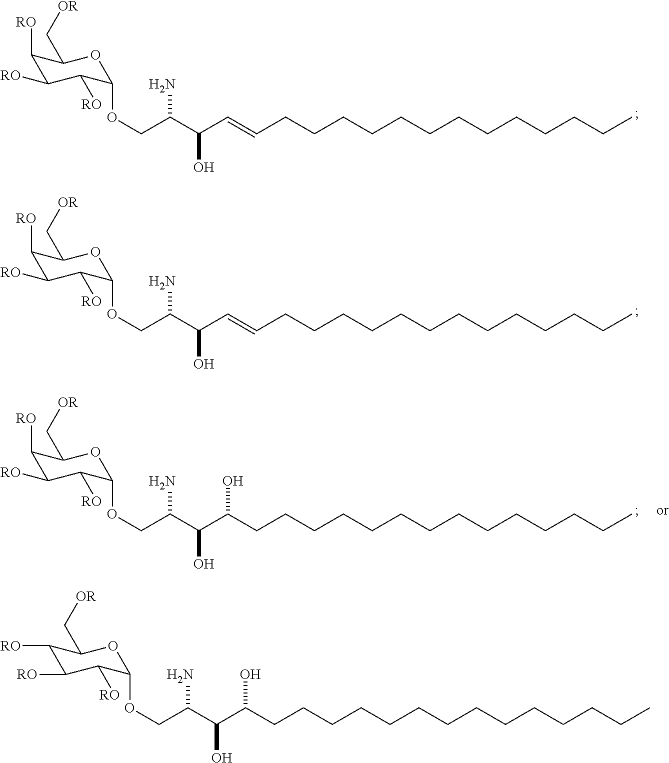
and R is independently —H, —OSO₃, or a pharmaceutically acceptable salt.

13. The composition of claim 10, wherein the compound is:

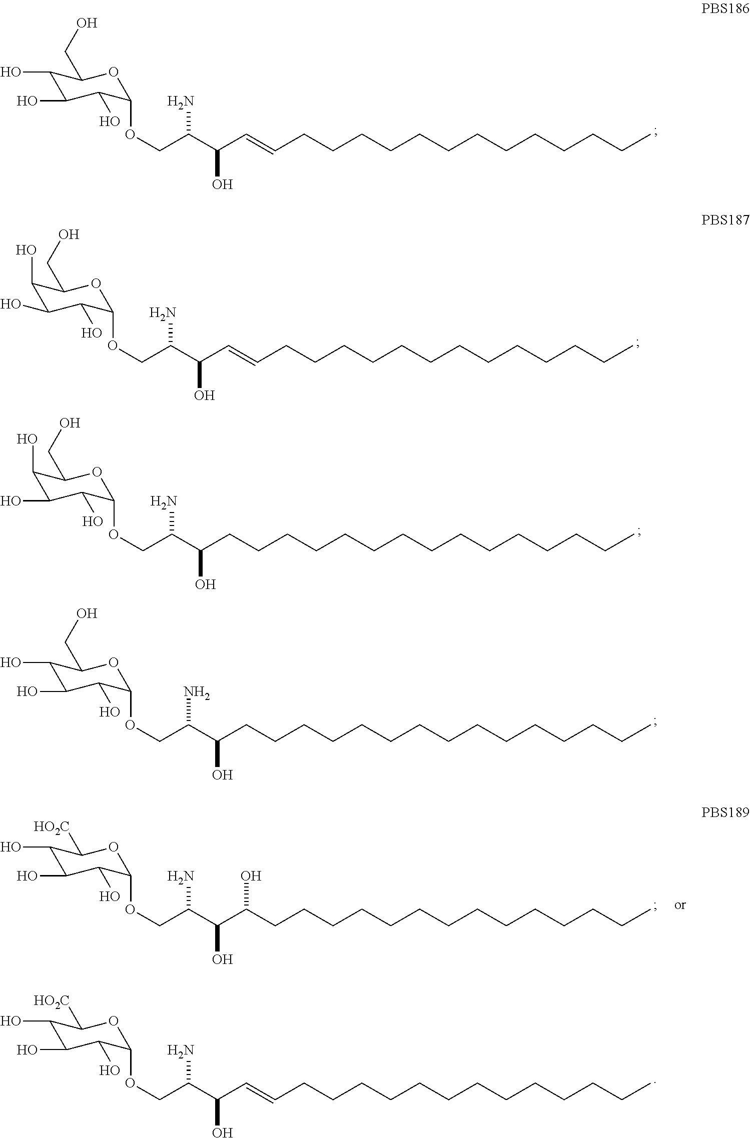 [SMILES: CCCCCCCCCCCCC/C=C/[C@@H](O)[C@@H](N)COC1OC(CO)C(O)C(O)C1O ; CCCCCCCCCCCCC/C=C/[C@@H](O)[C@@H](N)COC1OC(CO)C(O)C(O)C1O ; CCCCCCCCCCCCCCC[C@@H](O)[C@@H](N)COC1OC(CO)C(O)C(O)C1O ; CCCCCCCCCCCCCCC[C@@H](O)[C@@H](N)COC1OC(CO)C(O)C(O)C1O ; CCCCCCCCCCCCCC[C@@H](O)[C@@H](O)[C@@H](N)COC1OC(C(=O)O)C(O)C(O)C1O ; CCCCCCCCCCCCC/C=C/[C@@H](O)[C@@H](N)COC1OC(C(=O)O)C(O)C(O)C1O]

14. A composition comprising the compound of claim 5 and a physiologically acceptable vehicle.

15. The composition of claim 10, further comprising an antigen.

16. The composition of claim 15, wherein the antigen is a tumor antigen, a viral antigen, or a microbial antigen.

17. The composition of claim 15, wherein the composition is a vaccine.

18. A method of activating an NKT cell comprising contacting the NKT cell with a compound according to claim 1.

19. The method of claim 18, wherein the compound is bound to CD1d on an antigen presenting cell.

20. A method of inducing expression of α-glycosylceramide by an antigen presenting cell, comprising contacting the antigen presenting cell with an inhibitor of α-glycosidase or a ceramidase inhibitor.

21. A method of inducing NKT cell activation in a subject, comprising administering to a subject in need thereof a composition according to claim 10.

22. A method of inducing or enhancing NKT cell activation in a subject, comprising administering to a subject in need thereof an inhibitor of α-glycosidase or a ceramidase inhibitor.

23. A method of stimulating an immune response in a subject, comprising administering to a subject in need thereof:
a composition according to claim 10;
an NKT cell activated ex vivo by contacting the cell with a compound according to any one of claims 1 to 9; or
an antigen presenting cell comprising CD1d molecules loaded with a compound.

24. A method of stimulating an immune response in a subject, comprising administering to a subject in need thereof:
an inhibitor of α-glycosidase or a ceramidase inhibitor;
an NKT cell activated ex vivo by contacting the cell with an antigen presenting cell treated with an inhibitor of α-glycosidase or a ceramidase inhibitor; or
an antigen presenting cell treated with an inhibitor of α-glycosidase or a ceramidase inhibitor.

25. A method of modulating NKT cell activation in a subject, comprising administering to a subject in need thereof an antibody that binds α-galactosylceramide or α-glucosylceramide or an antibody that binds the complex formed by CD1d and α-galactosylceramide or α-glucosylceramide.

26. A method of treating an autoimmune disorder in a subject, comprising administering to a subject in need thereof an antibody that binds α-galactosylceramide or α-glucosylceramide or an antibody that binds the complex formed by CD1d and α-galatosylceramide or α-glucosylceramide.

27. The method of claim 26, wherein the autoimmune disorder is type I diabetes, rheumatoid arthritis, systemic lupus erythematosus, primary biliary cirrhosis, hepatitis, or multiple sclerosis.

28. A method of treating an allergic disorder in a subject, administering to a subject in need thereof an antibody that binds α-galactosylceramide or α-glucosylceramide or an antibody that binds the complex formed by CD1d and α-galatosylceramide or α-glucosylceramide.

29. The method of claim 28, wherein the allergic disorder is asthma, atopic dermatitis, eczema, or allergic rhinitis.

30. The method of claim 25, wherein the antibody is L317 or L363.

31. A method of treating cancer in a subject, comprising administering to a subject in need thereof a composition according to claim 10.

32. The method of claim 31, wherein the composition comprises a tumor antigen.

33. A method of treating a viral infection in a subject,
comprising administering to a subject in need thereof a composition according to claim 10.

34. The method of claim 33, wherein the composition comprises a viral antigen.

35. A method of treating a microbial infection in a subject, comprising administering to a subject in need thereof a composition according to claim 9.

36. The method of claim 35, wherein the composition comprises an antigen from a bacteria or parasite.

37. A method of identifying an NKT cell agonist, comprising
treating an antigen presenting cell with a candidate inhibitor of ceramidase or an α-glycosidase;
contacting an NKT cell with the treated antigen presenting cell; and
determining activation of the contacted NKT cell, wherein a candidate inhibitor that induces activation of the NKT cell is identified as an NKT cell agonist.

38. The method of claim 37, wherein the antigen presenting cells is a dendritic cell or a thymocyte.
